(19) United States
(12) Patent Application Publication
(10) Pub. No.: US 2025/0263364 A1
(43) Pub. Date: Aug. 21, 2025

(54) FORCED DYNAMIC OPERATION FOR ACRYLONITRILE MANUFACTURE

(71) Applicants: University of Virginia Patent Foundation, Charlottesville, VA (US); University of Houston, Houston, TX (US)

(72) Inventors: William Epling, Charlottesville, VA (US); Zhuoran Gan, Oak Ridge, TN (US); Lars Christian Grabow, Houston, TX (US); James Frank Brazdil, Leland, NC (US)

(21) Appl. No.: 19/052,377

(22) Filed: Feb. 13, 2025

Related U.S. Application Data
(60) Provisional application No. 63/555,954, filed on Feb. 21, 2024.

(51) Int. Cl. C07C 253/26; B01J 21/08; B01J 23/887

(52) U.S. Cl. CPC C07C 253/26; B01J 21/08; B01J 23/8876

(57) ABSTRACT

In one aspect, the disclosure relates to a process for acrylonitrile manufacture using forced dynamic operation over transition metal promoted bismuth molybdate-based catalysts. The forced dynamic operation leverages catalyst lattice oxygen in ammoxidation of propene to improved acrylonitrile productivity and yield. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application Ser. No. 63/555,954, filed Feb. 21, 2024, which is incorporated herein by reference in its entirety.

### STATEMENT REGARDING FEDERALLY SPONSORED RESEARCH OR DEVELOPMENT

This invention was made with government support under grant number DE-EE0009410 awarded by United States Department of Energy. The government has certain rights in the invention.

### BACKGROUND

The production of acrylonitrile by ammoxidation of propene has been commercialized for more than 60 years. This traditional process uses promoted bismuth molybdenum mixed-oxides (BMO) as a catalyst to react propene, ammonia, and oxygen to produce acrylonitrile. As a large volume commodity chemical useful in the production of carbon fiber and for other purposes, acrylonitrile has a production rate of around 7 million metric tons per year worldwide. With such high production capacity, the investment in infrastructure for the production of acrylonitrile utilizing steady-state operation (SSO) is large, which poses risk. The large-scale plants may not best cope with fluctuations in market demand.

Despite advances in acrylonitrile production research, there is still a scarcity of methods that are suitable for small-scale production. An ideal method would reduce or eliminate waste, would rely on decentralized plants in order to reduce transportation costs, and could quickly respond to changes in industrial demand. These needs and other needs are satisfied by the present disclosure.

### SUMMARY

In accordance with the purpose(s) of the present disclosure, as embodied and broadly described herein, the disclosure, in one aspect, relates to a process for acrylonitrile manufacture using forced dynamic operation over transition metal promoted bismuth molybdate-based catalysts. The forced dynamic operation leverages catalyst lattice oxygen in ammoxidation of propene to improved acrylonitrile productivity and yield.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a schematic diagram of an exemplary experimental set-up useful herein. FIG. 1B is a schematic of the 2-phase forced dynamic operation (FDO) strategy disclosed herein.

FIGS. 2A-2C are graphs showing (FIG. 2A) C₃H₆ conversion, (FIG. 2B) NH₃ conversion, and (FIG. 2C) ACN yield vs. temperature in steady-state operation (SSO) on Ni₈Fe₃-BMO, Co₈Fe₃-BMO and INEOS-BMO.

FIG. 3A is a graph of the acrylonitrile (ACN) concentration vs time of FDO with different duty cycles and SSO on Ni₈Fe₃-BMO at 330° C. FIG. 3B is a graph of the ACN production of FDO and SSO corresponding to FIG. 3A.

FIG. 4A is a graph of the ACN concentration vs time of FDO using varied O₂ concentrations in phase 2 and SSO on Ni₈Fe₃-BMO at 330° C. FIG. 4B is a graph of the ACN production of FDO and SSO corresponding to FIG. 4A.

FIGS. 5A-5B are graphs of ACN concentration vs time of FDO using the best scheme obtained in O₂ concentration effect investigations (O₂=100000 ppm) and SSO at (FIG. 5A) 360° C. and (FIG. 5B) 390° C. on Ni₈Fe₃-BMO. FIGS. 5C-5D are graphs of (FIG. 5C) ACN production and (FIG. 5D) ACN yield of FDO using the best scheme obtained in O₂ concentration effect investigations (O₂=100000 ppm) and SSO at 330° C., 360° C., and 390° C., respectively.

FIGS. 6A-6B are graphs illustrating ACN spike region (shaded area) used as an indicator of FDO performance of (FIG. 6A) Ni₈Fe₃-BMO and (FIG. 6B) INEOS-BMO. FIGS. 6C-6D are graphs showing the relationship between DOSC-C₃H₆ and (FIG. 6C) ACN production or (FIG. 6D) partial ACN production in ACN spike region on Examples 1-4 and Comparative Examples 1-6.

FIG. 7 is a graph showing the (left) XRD patterns of NiFe-BMO catalysts with varied Ni/Fe ratio; (right) enlarged XRD patterns of NiFe-BMO showing the evolution of Fe—Mo—O species.

FIG. 8 is a graph showing steady-state ACN production on the Ni₈Fe₃-BMO catalyst with varied O₂ concentration at 330° C.

FIGS. 9A-9D are graphs showing (FIGS. 9A-9B) FDO ACN concentration and production on Ni₈Fe₃-BMO at 330° C. with different cycle periods from 4-20 min with a 50% duty cycle, cycling between 3000 ppm and 9000 ppm O₂, and SSO at O₂=3000, 6000, 9000 ppm. (FIGS. 9C-9D) FDO results cycling between 3000 ppm and 10% O₂, and SSO ACN traces at O₂=3000 ppm, 5.15 and 10% are overlaid.

FIGS. 10A-10B show XRD characterization of Bi₂Mo₃O₁₂ and NiFe-BMO.

FIG. 11 shows BET surface area for various BMO catalysts. α-BMO stands for Bi₂Mo₃O₁₂; Ce-BMO, Co-BMO, Ni-BMO, Fe-BMO has the general formula of M₁Bi₁Mo₃O₁₂, M=Ce, Co, Ni, Fe; CoFe-BMO and NiFe-BMO has the general formula of M₈Fe₃Bi₁Mo₁₂Oₓ, M=Co, Ni.

FIGS. 12A-12B show surface imaging (He-ion microscopy) of BMO and SiO₂ supported BMO, respectively. BMO has the formula Bi₂Mo₃O_{12.}

FIG. 13 shows acrylonitrile production in μmol per hour with different BMO-containing catalysts doped with transition metals. The definitions of the BMOs are the same as those in FIG. 11.

FIG. 14 is a graph showing the (left) XRD patterns of NiF-BMO catalysts with varied Ni/Fe ratio; (right) enlarged XRD patterns of NiFe-BMO showing the evolution of Ni—Mo—O species.

FIG. 15A shows relative intensity of the signature phases of β-NiMoO4 and Fe₂Mo₃O₁₂ derived from XRD patterns of NiFe-BMO catalysts. FIG. 15B is a graph showing steady-state ACN production on the NiFe-BMO catalysts with varied Ni/Fe ratio at 330° C.

FIGS. 16A-16B show dynamic oxygen storage capacity (DOSC) for C₃H₆ using different BMO catalysts. α-BMO stands for Bi₂Mo₃O₁₂, NiFe-BMO stands for Ni₈Fe₃Bi₁₂Oₓ.

FIGS. 17A-17B show DOSC-C₃H₆ versus FDO ACN production and ACN spike for different BMO catalysts. α-BMO stands for Bi₂Mo₃O₁₂, Fe-BMO stands for FeBiMo₃O₁₂, Ni-BMO, stands for NiBiMo₃O₁₂, FeMO stands for Fe₂Mo₃O₁₂, NiMO stands for NiMoO₄, NiFe-BMO has the general formula of Ni₁₁₋ₐFeₐBiMo₁₂Oₓ, a=0, 0.5, 1, 3.

FIGS. 18A-18F show the effect of Ni/Fe ratio in NiFe-BMO. FIGS. 18A-18B show ACN concentration vs time using INEOS-BMO and Ni₈Fe₃-BMO in FDO with a 10 min cycle period, 50% duty cycle and in SSO at 330° C. Spike region is defined as the area where ACN production of FDO exceeds SSO as indicated with the shaded area. FIG. 18C shows ACN production in the spike region and the percentage of the spike region in total ACN production as a function of Ni/Fe ratio in NiₐFe₁₁₋ₐ-BMO, INEOS-BMO is listed as a reference. ACN production as a function of O₂ concentration in regeneration phase over INEOS-BMO, Ni_{10.5}Fe_{0.5}-BMO and Ni₈Fe₃-BMO using a 90% duty cycle 10 min FDO scheme. FDO condition is C₃H₆:NH₃:O₂=1500:1500:3000 ppm (reaction phase) and O₂ 3000 ppm (regeneration phase), balanced in N₂, total flow rate=50 sccm. SSO condition is C₃H₆:NH₃:O₂=1500:1500:3000 ppm, balanced in N₂, total flowrate=50 sccm, T=330° C.

FIGS. 19A-19G show the effects of lattice oxygen availability. FIG. 19A shows the protocol of reduction with C₃H₆+NH₃ followed by FDO on Ni₈Fe₃-BMO catalyst. 0.65 g catalyst was reduced for different time at 330° C. with C₃H₆:NH₃=1500:1500 ppm, total flow rate=50 sccm. The FDO scheme was 20 min cycle period with 50% duty cycle of C₃H₆:NH₃:O₂=1500:1500:3000 ppm (reaction phase) and O₂ 3000 ppm (regeneration phase), balanced in N₂, total flow rate=50 sccm. Before each reduction process the catalyst was pretreated in 10% O₂/N₂ 100 sccm at 500° C. for 1 hr. Between reduction and FDO, a 30 min N₂ purge was done to get rid of residual reductants. FIG. 19B shows the consumed lattice O₂ calculated based on O₂-containing products in the reduction process with different time. FIG. 19C shows images of catalyst bed after reduction for different time. FIG. 19D shows ACN concentration as a function of time over catalysts with different reduction time in FDO at 330° C. FIGS. 19E-19G show ACN moles formed, ACN yield and C₃H₆ conversion and product selectivity during the first FDO cycle indicated by the arrow in FIG. 19D. ACA=acetaldehyde, COₓ=CO+CO₂. FIG. 20 shows the effects of catalyst aging.

FIGS. 21A-21J show further effects of catalyst aging. In FIGS. 21A-21F, the samples were not pelletized. For FIGS. 21G-21J, the samples were pelletized.

FIGS. 22A-22B show structural changes in aged catalysts.

FIGS. 23A-23D show XPS analysis of FDO and SSO aged NiFe containing BMO samples.

FIGS. 24A-24F show the effect of N₂ flushing.

FIGS. 25A-25E show the effect of temperature changes.

FIGS. 26A-26B show the O₂ reaction order for fresh (FIG. 26A) and aged (FIG. 26B) catalyst.

FIGS. 27A-27B show the effect of aging on the disclosed catalysts relative to a commercial catalyst.

FIGS. 28A-28B show the effect of temperature on the disclosed catalysts (FIG. 28A) relative to a commercial catalyst (FIG. 28B).

FIGS. 29A-29C show XRD of referenced mixed MoOx detected in the disclosed catalyst formulations.

FIGS. 30A-30B show XRD of Ni-BMO and Fe-BMO, respectively, demonstrating presence of α-BMO in Ni-BMO and Fe-BMO.

FIGS. 31A-31D show, respectively, C conversion, N conversion, ACO yield, and ACN yield for NiFe-BMO and CoFe-BMO relative to a commercial catalyst sample.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In one aspect, the present disclosure relates to the manufacture of acrylonitrile (ACN) using forced dynamic operation (FDO) in the ammoxidation of propene over transition metal promoted bismuth molybdate-based catalysts.

Forced dynamic operation (FDO), or periodic operation of a reactor, is a technique that involves periodically changing reactor inputs or the reactor environment. In one aspect, in FDO, one or more reactant concentrations to the reactor are purposefully varied as a function of time, or the reactor or reactant gas mixture temperature are varied as a function of time, or the reactor pressure is varied as a function of time. This can also be called modulation, cycling, or periodic forcing. In one aspect, the goals of FDO include improving selectivity and yield of the desired product and/or enhancing reaction rate. In another aspect, FDO can be used to regenerate a catalyst, such that activity can be re-attained. In a further aspect, one method of FDO is realized through composition forcing or feed modulation, e.g., step-changes in reactor inlet concentration in a periodic manner. FDO has proven advantageous in many reactions such as NH₃ synthesis, methane combustion, and especially, partial oxidation of hydrocarbons. In an aspect, in partial oxidation reactions, oxygen in the catalyst or lattice oxygen plays a major role, in that the desired product is selectively produced, not becoming completely oxidized to H₂O and CO₂. Further in this aspect, the catalyst functions as an oxygen carrier allowing the partial oxidation reaction to be divided into two separate steps: reduction of the catalyst by hydrocarbon and NH₃ in one step and re-oxidation by gaseous O₂ in another step. In one aspect, such a process is perfectly aligned with FDO with feed modulation.

In another aspect, some promoted BMO catalysts have been developed for ammoxidation of propene and show high yield towards ACN in steady-state operation (SSO) processes. In one aspect, disclosed herein is a method for producing BMO-based catalyst formulations that are efficient for FDO, which can provide improved ACN productivity and/or yield compared to SSO. As defined herein, steady-state operation (SSO) refers to continuous operation in the presence of a catalyst for at least about one hour under approximately constant feed composition, temperature, pressure and other process conditions. In some aspects, SSO can refer to other time periods, such as continuous operation in the presence of a catalyst for at least about 10 min, 20 min, 30 min, 40 min, 50 min, 1 hour, 1.25 hours, 1.5 hours, or 2 hours. In a further aspect, the FDO parameters can be tuned for a catalyst, or a catalyst tuned for the parameters. In one aspect, FDO aged catalyst retains performance and properties closer to fresh catalyst as opposed to SSO aged catalyst, thus prolonging the useful lifetime of the catalyst. In another aspect, FDO can have lower energy requirements than SSO. In still another aspect, FDO offers additional advantages versus SSO including, but not limited to substantial variable manufacturing cost reduction and enhanced process efficiency due to (i) increased yield and selectivity to acrylonitrile, (ii) reduced raw material usage for propene and ammonia feedstocks, and (iii) increased operational time interval for catalyst change-out due to enhanced catalyst lifetime. In some aspects, NiFe and/or CoFe promoted BMO show better acrylonitrile production under FDO. In an aspect, during SSO, the rate of catalyst reduction and reoxidation must be equal. Further in this aspect, FDO has performance benefits over SSO, because the catalyst reduction and reoxidation occur in different reaction environments, which can be independently optimized to increase the rate of reduction or oxidation. As the rate of reduction and oxidation is an intrinsic catalyst property, the catalyst composition can be tuned to favor reduction or oxidation reactions. In one aspect, and without wishing to be bound by theory, for best overall performance, the catalyst composition must produce rates of catalyst reduction and oxidation that match the periods of the reduction and oxidation cycle of FDO. The rate of catalyst reduction and oxidation, can be approximately quantified by measuring its dynamic oxygen storage capacity (DOSC). In another aspect, practical benefits of FDO over SSO include temporal separation of the oxidant (O₂) from the hydrocarbon and NH₃, which greatly expands the accessible reductant-to-oxidant ratios and allows safe operation within the flammability limit of the overall feed mixture.

In one aspect, disclosed herein is a method for manufacturing an acrylic compound or intermediate, the method including alternately flowing a first gas phase and a second gas phase over a catalyst in a reactor; wherein the first gas phase, the second gas phase, or both include propene, ammonia, and oxygen; wherein the catalyst includes a transition metal promoted bismuth molybdate catalyst; wherein at least one reactor input or condition is modified periodically using forced dynamic operation (FDO); and wherein the acrylic compound or intermediate is selected from acrylonitrile, acrolein, acrylic acid, or any combination thereof.

In another aspect, the at least one reactor input can be composition of the first gas phase, composition of the second gas phase, flow rate of the first gas phase, flow rate of the second gas phase, duty cycle of the reactor, reaction temperature, cycle period of the reactor, or any combination thereof. In one aspect, the flow rate of the first gas phase and the flow rate of the second gas phase can be the same. In another aspect, the cycle period of the reactor can be from about 0.1 min to about 2 hours, or can be about 0.1, 0.5, 1, 2, 3, 4, 5, 6, 7, 8, 9, 10, 11, 12, 13, 14, 15, 16, 17, 18, 19, or about 20 min, or can be about 0.5, 1, 1.5, or about 2 hours, or a combination of any of the foregoing values, or a range encompassing any of the foregoing values. In another aspect, the duty cycle of the reactor can be from about 50% to about 95%, or can be about 50, 55, 60, 65, 70, 75, 80, 85, 90, or about 95%, or a combination of any of the foregoing values, or a range encompassing any of the foregoing values. In one aspect, the reaction temperature can be from about 300° C. to about 500° C., or can be about 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or about 500° C., or can be from about 320° C. to about 450° C., or a combination of any of the foregoing values, or a range encompassing any of the foregoing values.

In one aspect, the FDO strategy consists of at least two alternating phases, but can consist of three or more phases in other aspects. In one example of a two phase system, one phase has a propene:ammonia:oxygen ratio of 1:1:2, while the other phase consists of O₂ only. In some aspects, O₂ can be provided as substantially pure O₂, or can be provided as an oxygen-containing gas or mixed gas composition such as, for example, air. Further in this aspect, the O₂ concentration in phase 2 is in the range of the equivalent of from about 0.5 to about 67 times the propene concentration in phase 1, or can be about 0.5, 1, 2, 5, 10, 15, 20, 25, 30, 35, 40, 45, 50, 55, 60, 65, or about 67 times the propene concentration in phase one, or a combination of any of the foregoing values, or a range encompassing any of the foregoing values. In yet another aspect, the feed mixtures are all balanced by an inert gas such as, for example, N₂, in the two or three or more phases.

In another aspect, the FDO strategy consists of 2 alternating phases; one phase has a propene:ammonia:oxygen ratio of 1:1:2, while the other phase has a propene:ammonia:oxygen ratio of from about 1:1:2 to 1:1:80 or from about 1:1:6 to 1:1:67.

In an aspect, the catalyst for ammoxidation of propene in FDO, includes a support material such as a silica support and a mixed-oxide complex having the following formula:

MₐFe_{b}Bi_{c}Mo₁₂Oₓ

wherein M can be selected nickel, cobalt, manganese, magnesium, zinc, chromium, or any combination thereof. In one aspect, M is Ni. In another aspect, 0<a+b≤11, or 5<a+b≤11, or a+b=1. Further in this aspect, a or b can be 0. In an alternative aspect, a can be from about 0.1 to about 10.9 and b can be from about 0.1 to about 10.9. In a further aspect, 0<c<3, or 1<c<2, or c is about 1. In still another aspect, x can be the total oxygen number that meets the requirement of the valence of the elements. In one aspect, the catalyst can be Ni₁₁Fe₀Bi₁Mo₁₂Oₓ, Ni_{10.5}Fe_{0.5}Bi₁Mo₁₂Oₓ, Ni₁₀Fe₁Bi₁Mo₁₂Oₓ, Ni₈Fe₃Bi₁Mo₁₂Oₓ, or any combination thereof.

In one aspect, the catalyst includes a support material such as, for example, a silica support. Further in this aspect, the content of the support material can be from about 20 wt % to about 80 wt %, or can be 20, 25, 30, 35, 40, 45, 50, 55, 60, 65, 70, 75, or about 80 wt %, of a combination of any of the foregoing values, or a range encompassing any of the foregoing values.

In an aspect, provided herein is a process for low-volume, de-centralized acrylonitrile manufacture. However, in another aspect, the current disclosure is also applicable to the production of acrolein (ACO) by the partial oxygenation of propene, or further to acrylic acid production.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a BMO catalyst,” “a metal,” or “a gas,” include, but are not limited to, mixtures or combinations of two or more such BMO catalysts, metals, or gases, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a feed gas refers to an amount that is sufficient to achieve the desired improvement in the property modulated by the formulation component, e.g. achieving the desired level of conversion of starting materials to acrylonitrile. The specific level in terms of wt % in a composition required as an effective amount will depend upon a variety of factors including the identities and amounts of other feed or carrier gases present, scale of the reaction, temperature and pressure of the reaction, and the like.

As used herein, the terms “optional” or “optionally” means that the subsequently described event or circumstance can or cannot occur, and that the description includes instances where said event or circumstance occurs and instances where it does not.

A “transition metal promoted bismuth molybdate catalyst” as used herein is a catalyst containing bismuth and molybdate ions in its crystal lattice alongside one or more transition metals including, but not limited to, Fe, Ni, Co, Mn, Zn, Cr, and combinations thereof. In some aspects, a transition metal promoted bismuth molybdate catalyst can also include an alkali metal and/or an alkaline earth metal such as, for example, Mg, as long as it also contains at least one transition metal. As used herein and as defined by convention by the International Union of Pure and Applied Chemistry (IUPAC), the term “transition metal” refers to one or more elements in Groups 3 to 12, inclusive, in the Periodic Table of Elements.

Unless otherwise specified, temperatures referred to herein are measured at atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A method for manufacturing an acrylic compound or intermediate, the method comprising alternately flowing a first gas phase and a second gas phase over a catalyst in a reactor;
wherein the first gas phase, the second gas phase, or both comprise propene, ammonia, and oxygen;
wherein the catalyst comprises a transition metal promoted bismuth molybdate catalyst;
wherein at least one reactor input or condition is modified periodically using forced dynamic operation (FDO); and
wherein the acrylic compound or intermediate comprises acrylonitrile, acrolein, acrylic acid, or any combination thereof.

Aspect 2. The method of aspect 1, wherein the oxygen is provided as substantially pure oxygen gas.

Aspect 3. The method of aspect 1, wherein the oxygen is provided as a component of a mixed gas composition.

Aspect 4. The method of aspect 3, wherein the mixed gas composition comprises air.

Aspect 5. The method of any one of aspects 1-4, further comprising a third gas phase.

Aspect 6. The method of aspect 5, wherein the third gas phase comprises propene, ammonia, oxygen, or any combination thereof.

Aspect 7. The method of any one of aspects 1-6, wherein the at least one reactor input comprises composition of the first gas phase, composition of the second gas phase, flow rate of the first gas phase, flow rate of the second gas phase, duty cycle of the reactor, reaction temperature, cycle period of the reactor, or any combination thereof.

Aspect 8. The method of any one of aspects 1-7, wherein the first gas phase comprises propene, ammonia, and oxygen in a molar ratio of about 1:1:2.

Aspect 9. The method of any one of aspects 1-8, wherein the second gas phase is selected from oxygen or a combination of propene, ammonia, and oxygen.

Aspect 10. The method of aspect 9, wherein the second gas phase is propene, ammonia, and oxygen in a molar ratio of from about 1:1:6 to about 1:1:67.

Aspect 11. The method of aspect 9, wherein the second gas phase is oxygen and wherein the oxygen is present in a molar amount of from about 0.5 to about 67 times a molar amount of propene in the first gas phase.

Aspect 12. The method of any one of aspects 1-11, wherein the first gas phase, the second gas phase, and, if present, the third gas phase, or any combination thereof further comprise an inert gas.

Aspect 13. The method of aspect 12, wherein the inert gas comprises N₂.

Aspect 14. The method of any one of aspects 7-13, wherein the flow rate of the first gas phase and the flow rate of the second gas phase are the same.

Aspect 15. The method of any one of aspects 7-14, wherein the cycle period of the reactor is from about 0.1 min to about 2 hours.

Aspect 16. The method of any one of aspects 7-15, wherein the duty cycle of the reactor is from about 50% to about 95%.

Aspect 17. The method of any one of aspects 7-16, wherein the reaction temperature is from about 300° C. to about 500° C.

Aspect 18. The method of any one of aspects 1-17, wherein the catalyst has a formula MₐFe_{b}Bi_{c}Mo₁₂Oₓ, wherein M comprises a transition metal, an alkaline earth metal, an alkali metal, or any combination thereof;
wherein 0<a+b≤11;
wherein 0<c<3; and
wherein x has a value such that an amount of oxygen in the catalyst balances a charge of M, Fe, Bi, and Mo combined.

Aspect 19. The method of aspect 18, wherein a is from 0.1 to 10.9.

Aspect 20. The method of aspect 18 or 19, wherein b is from 0.1 to 10.9.

Aspect 21. The method of any one of aspects 18-20, wherein M comprises Ni, Co, Mn, Mg, Zn, Cr, or any combination thereof.

Aspect 22. The method of aspect 21, wherein M is Ni.

Aspect 23. The method of any one of aspects 18-22, wherein the catalyst comprises Ni₁₁Fe₀Bi₁Mo₁₂Oₓ, Ni_{10.5}Fe_{0.5}Bi₁Mo₁₂Oₓ, Ni₁₀Fe₁Bi₁Mo₁₂Oₓ, Ni₈Fe₃Bi₁Mo₁₂Oₓ, or any combination thereof.

Aspect 24. The method of any one of aspects 1-23, wherein the catalyst further comprises a support material.

Aspect 25. The method of aspect 24 wherein the support material comprises silica.

Aspect 26. The method of aspect 24 or 25, wherein the support material is present at from about 20 wt % to about 80 wt % of the catalyst.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Ni₈Fe₃Bi₁Mo₁₂O₅₀+20 wt % SiO_{2.}

Solution A was prepared by dissolving 0.69 g Bi(NO₃)₃·5H₂O into 10 ml diluted HNO₃ solution (3 ml 70% HNO₃ mixed with 7 ml deionized water).

Solution B was prepared by dissolving 1.71 g Fe(NO₃)₃·9H₂O into 100 ml deionized water, then adding 3.29 g Ni(NO₃)₂·6H₂O.

Solution C was prepared by dissolving 3 g (NH₄)₆Mo₇O₂₄·4H₂O into 50 ml deionized water. 2.94 g 34 wt % sodium-free silica sol having a SiO₂ particle diameter of 22 nm was added to the resulting solution.

Solution B was mixed with solution A, then solution C was added to B dropwise then heated to 80° C. with 250 rpm stirring overnight. The resulting slurry was heated for another 6 h at 120° C. to dryness.

The precursor solid was collected and calcined in air at 290° C. for 3 h, then 425° C. for 3 h, then 560° C. for 3 h, with a ramping rate between temperatures of 5° C./min. The catalyst is denoted as Ni₈Fe₃-BMO.

### Example 2: Ni₁₀Fe₁Bi₁Mo₁₂O₄₉+20 wt % SiO₂

The catalyst was prepared following the procedure in Example 1, where the amount of Bi(NO₃)₃·5H₂O 0.7 g, that of Fe(NO₃)₂·9H₂O was 0.58 g, that of Ni(NO₃)₂·6H₂O was 4.17 g, that of (NH₄)₆Mo₇O₂₄·4H₂O was 3.04 g, with others being the same as Example 1. The catalyst is denoted as Ni₁₀Fe₁-BMO.

### Example 3: Ni_{10.5}Fe_{0.5}Bi₁Mo₁₂O₅₀+20 wt % SiO₂

The catalyst was prepared following the procedure in Example 1, where the amount of Bi(NO₃)₃·5H₂O 0.7 g, that of Fe(NO₃)₂·9H₂O was 0.29 g, that of Ni(NO₃)₂·6H₂O was 4.39 g, that of (NH₄)₆Mo₇O₂₄·4H₂O was 3.04 g, with others being the same as Example 1. The catalyst is denoted as Ni_{10.5}Fe_{0.5}-BMO.

### Example 4: Ni₁₁Fe₀Bi₁Mo₁₂O+20 wt % SiO₂

The catalyst was prepared following the procedure in Example 1, where the amount of Bi(NO₃)₃·5H₂O 0.7 g, that of Fe(NO₃)₂·9H₂O was 0, that of Ni(NO₃)₂·6H₂O was 4.6 g, that of (NH₄)₆Mo₇O₂₄·4H₂O was 3.05 g, with others being the same as Example 1. The catalyst is denoted as Ni₁₁Fe₀-BMO.

### Example 5: Co₈Fe₃Bi₁Mo₁₂O₅₀+20 wt % SiO₂

The catalyst was prepared following the procedure in Example 1, where the amount of Bi(NO₃)₃·5H₂O 0.69 g, that of Fe(NO₃)₂·9H₂O was 1.73 g, that of (NH₄)₆Mo₇O₂₄·4H₂O was 3.03 g, 3.33 g Co(NO₃)2·6H₂O was added to solution B, with others being the same as Example 1. The catalyst is denoted as Co₈Fe₃-BMO.

### Comparative Example 1: Bi₂Mo₃O₁₂+20 wt % SiO₂

Solution A was prepared by dissolving 9.6 g Bi(NO₃)₃·5H₂O into 0.2 M citric acid (CA) solution with ratio 1 Bi:1 CA, then adding 12.6 ml nitric acid (NA), with 10 NA:1 CA.

Solution B was prepared by dissolving 5.3 g (NH₄)₆Mo₇O₂₄·4H₂O into 0.3 M CA solution, with ratio 1 Mo:1 CA, then adding 6.5 g 34 wt % sodium-free silica sol having a SiO₂ particle diameter of 22 nm.

Solution B was slowly added to solution A while heated to 80° C. with stirring at 250 rpm overnight, the slurry was further heated to 120° C. for 6 h to dryness.

The resulting solid was grinded and calcined in air at 500° C. for 6 h with a ramping rate to said temperature of 5° C./min. The catalyst is denoted as α-BMO.

### Comparative Example 2: Ni₁Mo₁O₄+20 wt % SiO₂

3.23 g (NH₄)₆Mo₇O₂₄·4H₂O was dissolved into 200 ml deionized water, then 5.32 g Ni(NO₃)₂·6H₂O was added, and then 2.94 g 34 wt % sodium-free silica sol having a SiO₂ particle diameter of 22 nm was added to the resulting solution. The heating and calcination procedures were the same as Example 1. The catalyst is denoted as NiMO.

### Comparative Example 3: Fe₂Mo₃O₁₂+20 wt % SiO₂

The catalyst was prepared following the procedure in Comparative example 2, where the amount of (NH₄)₆Mo₇O₂₄·4H₂O was 3.27 g, that of Fe(NO₃)₃·9H₂O was 5 g, with the others being the same as Comparative example 2. The catalyst is denoted as FeMO.

### Comparative Example 4: Ni₁Bi₁Mo₃O_{11.5}+20 wt % SiO₂

The catalyst was prepared following the procedure in Example 1, where the amount of Bi(NO₃)₃·5H₂O 2.62 g, that of Ni(NO₃)₂·6H₂O was 1.6 g, that of (NH₄)₆Mo₇O₂₄·4H₂O was 2.86 g, with others being the same as Example 1 except no Fe precursor was added. The catalyst is denoted as Ni-BMO.

### Comparative Example 5: Fe₁Bi₁Mo₃O₁₂+20 wt % SiO₂

The catalyst was prepared following the procedure in Example 1, where the amount of Bi(NO₃)₃·5H₂O 2.61 g, that of Fe(NO₃)₂·9H₂O was 2.17 g, that of (NH₄)₆Mo₇O₂₄·4H₂O was 2.85 g, with others being the same as Example 1 except no Ni precursor was added. The catalyst is denoted as Fe-BMO.

### Comparative Example 6: INEOS-BMO

A commercial silica supported multi-component (including but not limited to Ni, Fe, Cr) promoted bismuth molybdate-based catalyst was provided by INEOS Nitriles USA LLC (Lima, Ohio). The catalyst is denoted as INEOS-BMO.

### Example 7: Characterization and Yield

X-ray diffraction (XRD) was carried out with a PANalytical Empyrean X-ray diffractometer using Cu Kα radiation operated at 40 kV and 40 mA. Dynamic oxygen storage capacity measurements were performed using C₃H₆ as a probe molecule, with alternating exposures of C₃H₆:N₂:O₂:N₂=2 min:3 min:2 min:3 min, C₃H₆=1500 ppm and O₂=3000 ppm, total flowrate=300 sccm, T=330° C.

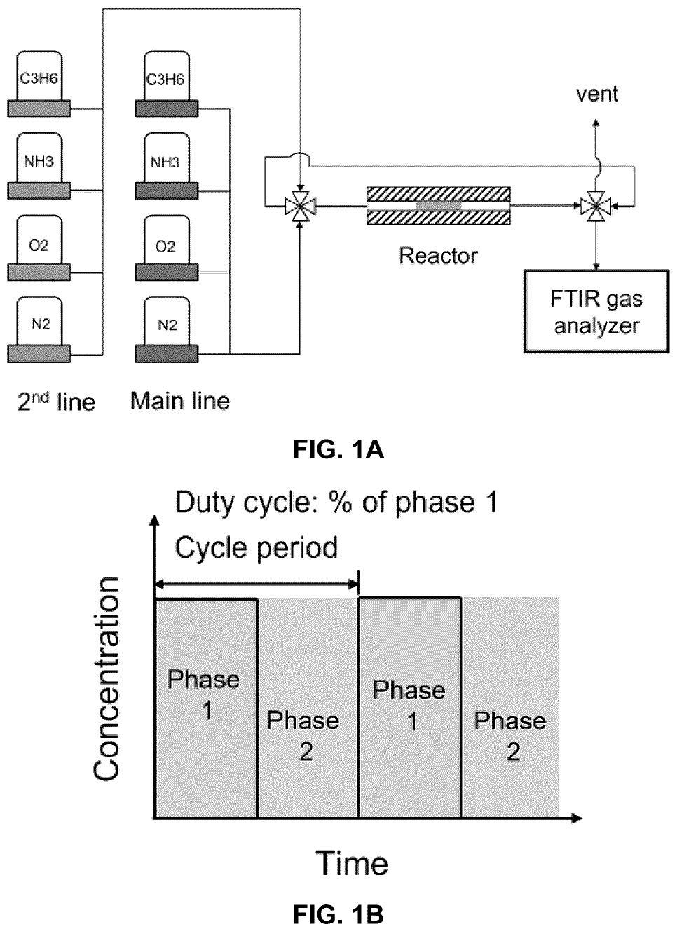

Catalytic performance measurements. The propene ammoxidation reaction experiments were performed in a horizontal quartz tube reactor with an internal diameter of 11 mm. The quartz tube reactor resided in a Thermo Scientific Lindberg Blue M electric furnace. The catalyst was pelletized and sieved to 40-60 mesh (250-425 μm) pellets, mixed with nitric acid washed SiO₂ of the same size and placed between two quartz wool plugs. Two K-type thermocouples were placed at the end of the two quartz wool plugs to measure the temperature upstream and downstream of the catalyst. Forced dynamic operation was achieved by the set-up illustrated in FIG. 1A, where two separate gas lines each can deliver C₃H₆, NH₃, O₂, and N₂ (carrier gas), the flowrates are regulated by MKS mass flow controllers. The inlet of the reactor was regulated by an automated 4-way valve, which alternates the incoming gas mixture. A two-phase FDO scheme was usually applied throughout this disclosure, where feed gases in phase 1 corresponding to the main line, feed gases in phase 2 corresponding to the 2^{nd} line. When in FDO mode, the gas mixture in one line is going through the reactor, and the outlet concentrations are measured using a MKS MG2030 FTIR gas analyzer. The gas mixture in the other line is going through the bypass line then vented to an exhaust hood. After a certain time, i.e., when a phase is over, the 4-way valve turns, and another phase is begun, the process alternates periodically. The time combining phase 1 and phase 2 is called the cycle period, and the percentage of phase 1 in the cycle period is called the duty cycle, shown in FIG. 1B. The propene conversion, ammonia conversion, ACN selectivity, ACN yield are calculated as follows:

$$\begin{matrix}{C}_{3} {H}_{6} Conversion = \frac{moles of {C}_{3} {H}_{6 in} - moles of {C}_{3} {H}_{6 out}}{moles of {C}_{3} {H}_{6 in}} \times 100 \% \\ {NH}_{3} Conversion = \frac{moles of {NH}_{3 in} - moles of {NH}_{3 out}}{moles of {NH}_{3 in}} \times 100 \% \\ ACN selectivity = \frac{3 \times moles of ACN}{3 \times moles of {C}_{3 formed} + 2 \times moles of {C}_{2 formed} + moles of {C}_{1 formed}} \times 100 \% \\ ACN yield = {C}_{3} {H}_{6} Conversion \times ACN selectivity \times 100 \%\end{matrix}$$

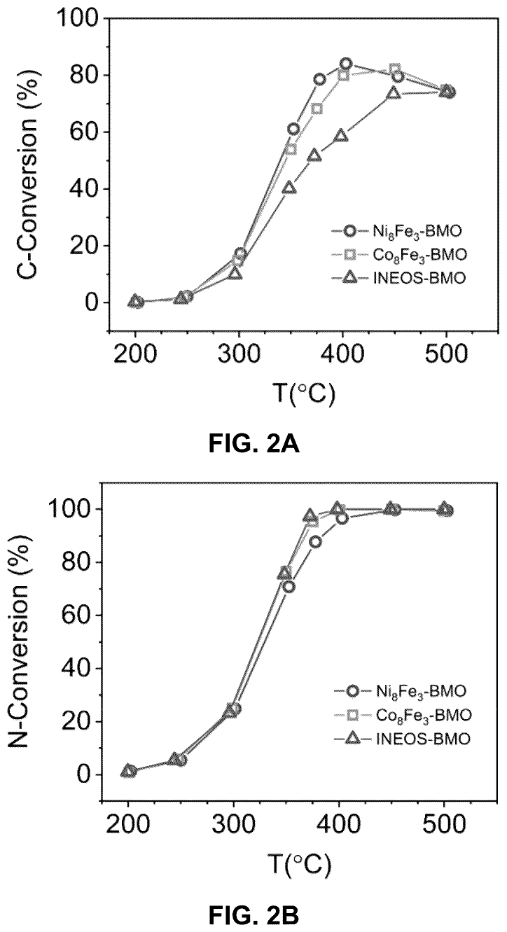

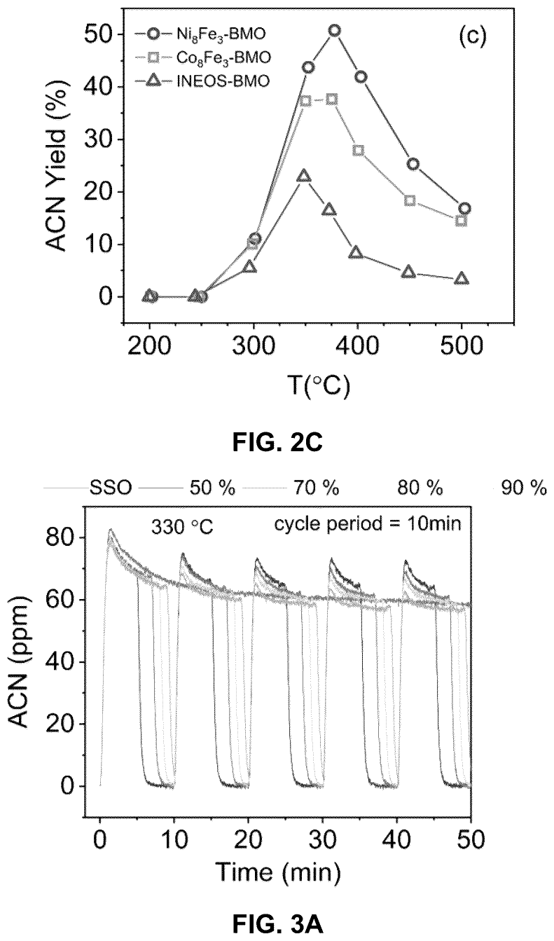

The catalyst was pretreated with 10% O₂/N₂ at 500° C. for 1 h in a flowrate of 100 sccm before reaction. In disclosed process, the total flowrate in the reactor is usually 50 sccm. This results in a contact time=2.3 s. Due to the constraints of the FTIR analyzer (the inlet flowrate needs to be >200 sccm), the FTIR inlet is diluted with additional 250 sccm N₂ flow, making the total flowrate 300 sccm. Thus, the gas concentrations reflected in the figures are diluted 6 times (to obtain the real concentrations, the values plotted should be multiplied by 6). A series of temperature programmed reactions were done from 200-500° C. using Ni₈Fe₃-BMO, Co₈Fe₃-BMO, and INEOS-BMO to evaluate the catalytic performance at steady-state. The feed conditions were, C₃H₆:NH₃:O₂=1500 ppm:1500 ppm:3000 ppm, with balance N₂, total flowrate=50 sccm, C₃H₆ GHSV=0.65 h⁻¹. The ramping rate between temperatures was 5° C./min and the dwell time is 2 h at each temperature. The results are shown in FIGS. 2A-2C. The Ni₈Fe₃-BMO sample exhibits the best ACN yield (50%) at 375° C. amongst the three catalysts, although not as high as those of some reported bismuth molybdate-based catalysts showing >80% ACN yield. The emphasis of the present disclosure is the performance under FDO modes, which will be shown in the following examples.

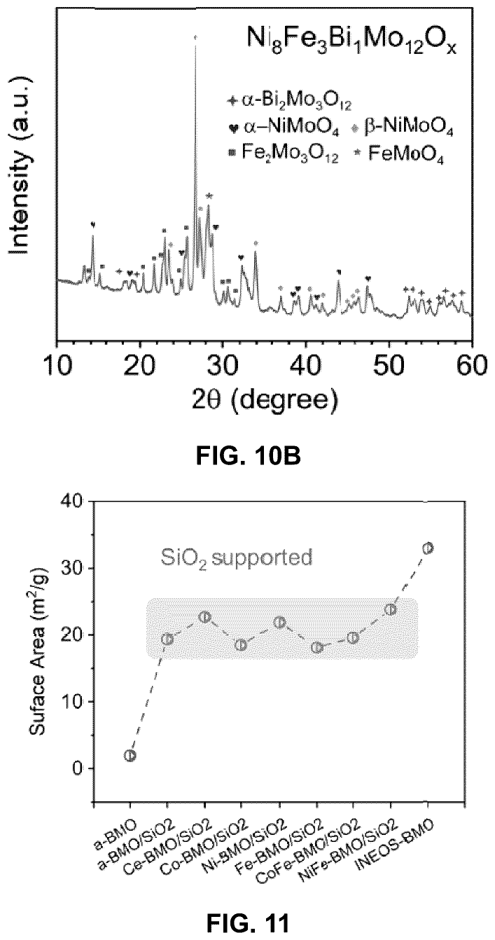

FIGS. 10A-10B show XRD characterization of Bi₂Mo₃O₁₂ and NiFe-BMO. Bi₂Mo₃O₁₂ contains mainly α-BMO, while NiFe-BMO contains at least α-BMO, NiMoOx, and FeMoOx.

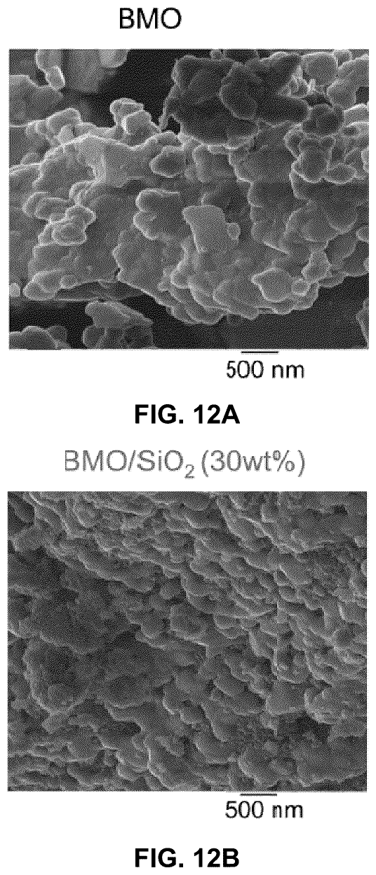

FIG. 11 shows Brunauer-Emmett-Teller (BET) surface area for various BMO catalysts. FIGS. 12A-12B show surface imaging (He-ion microscopy) of BMO and SiO₂ supported BMO, respectively. The presence of larger particles may correspond to bismuth molybdenum, while the smaller particles are indicative of silica. Samples in BMO appear more aggregated compared to BMO/SiO₂ with Si dispersion in between.

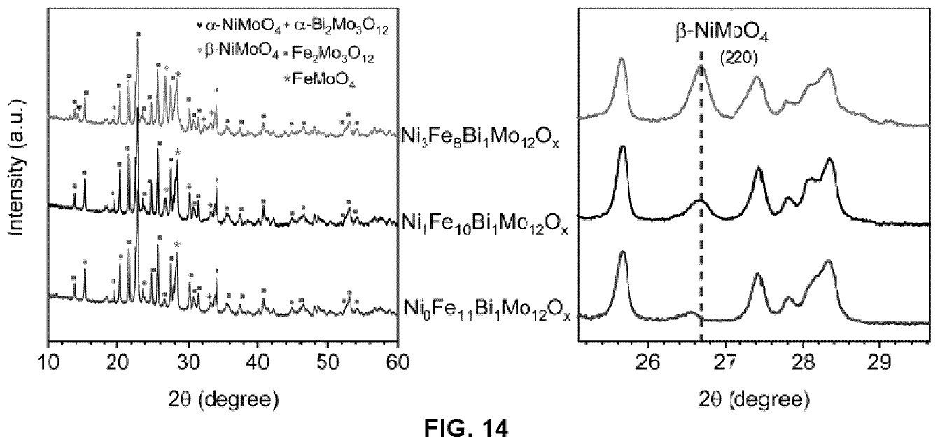

FIG. 14 is a graph showing the (left) XRD patterns of NiFe-BMO catalysts with varied Ni/Fe ratio; (right) enlarged XRD patterns of NiFe-BMO showing the evolution of Ni—Mo—O species. NiMo features grow more pronounced with increasing nickel content.

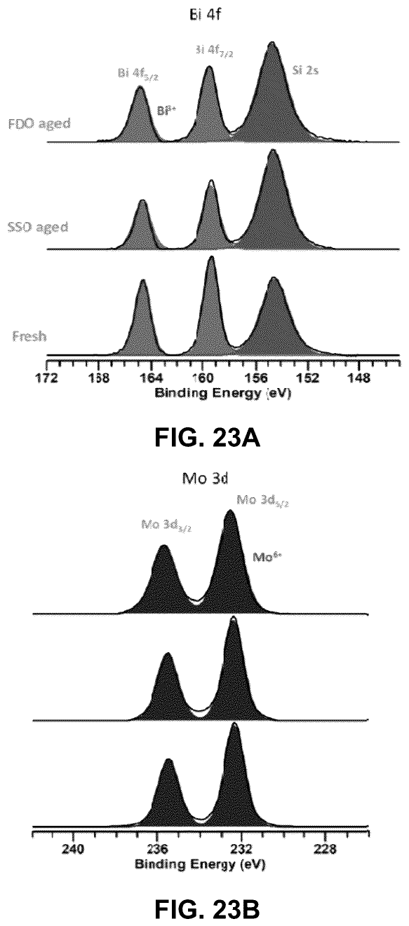

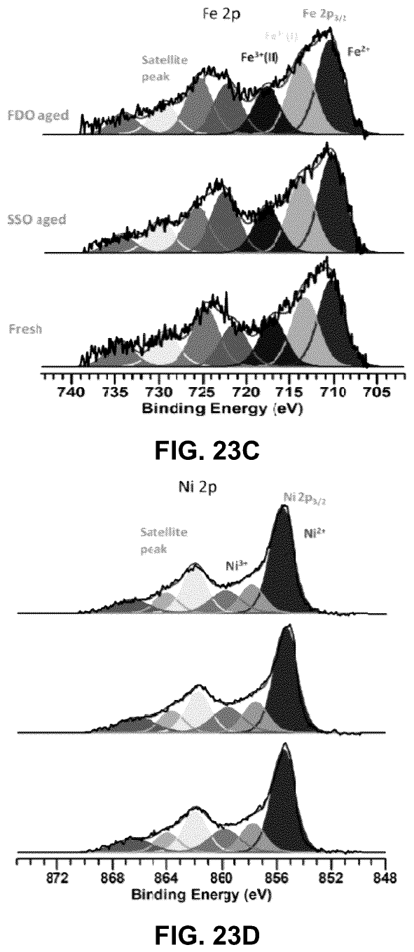

FIGS. 23A-23D show XPS analysis of FDO and SSO aged NiFe containing BMO samples. Aged samples were evaluated against the baseline spectra of fresh NiFe-BMO. XPS analysis of the aged samples reveals no reduction in Bi and Mo regions. Consistent with earlier reports, Ni is present as Ni²⁺ and Ni³⁺, while iron exists as Fe²⁺ and two distinct configurations of Fe³⁺. The oxidation states of Ni and Fe stay consistent after aging, though iron seemed to have migrated to the surface in the aged samples. See also Table 1 below:

TABLE 1
Fe³⁺/Fe²⁺ and Ni³⁺/Ni²⁺ Ratios

| Sample | Fe³⁺/Fe²⁺ | Ni³⁺/Ni²⁺ |
|---|---|---|
| Fresh | 0.791 | 0.68 |
| SSO Aged | 0.827 | 0.75 |
| FDO Aged | 0.774 | 0.626 |

Quantification from the XPS scans is presented in Tables 2 and 3 below. This quantification is derived from the average of two-point scans.

TABLE 2
Elemental Analysis of NiFeBMO Samples

| Sample | Bi | Mo | Si | Ni | Fe | O | C |
|---|---|---|---|---|---|---|---|
| Fresh | 1.26 | 7.9 | 14.45 | 5.56 | 2.75 | 44.28 | 23.78 |
| FDO (aged) | 0.59 | 9.125 | 18.9 | 6.265 | 3.685 | 54.61 | 5.415 |
| SSO (aged) | 0.86 | 8.48 | 18.79 | 5.58 | 4.065 | 53.8 | 8.415 |

TABLE 3
Bismuth Ratios in NiFeBMO Samples

| NiFeBMO | Bi/Mo | Bi/Ni | Bi/Fe |
|---|---|---|---|
| Fresh | 0.159 | 0.226 | 0.458 |
| FDO (aged) | 0.108 | 0.158 | 0.268 |
| SSO (aged) | 0.101 | 0.154 | 0.211 |

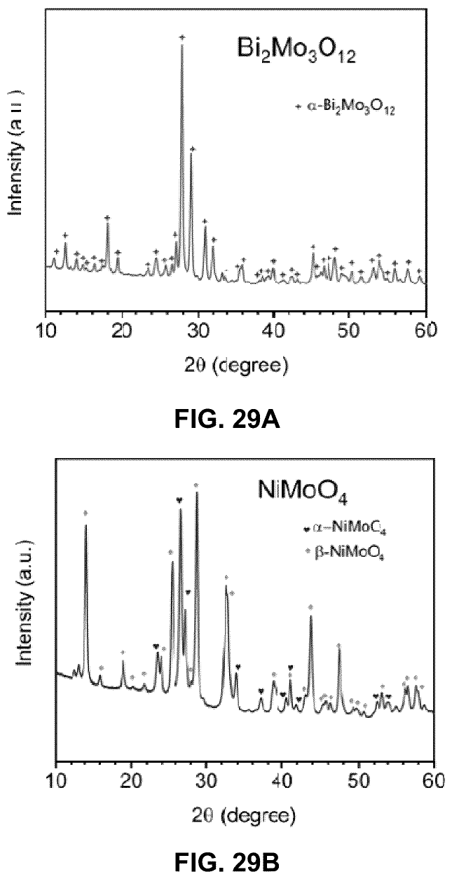

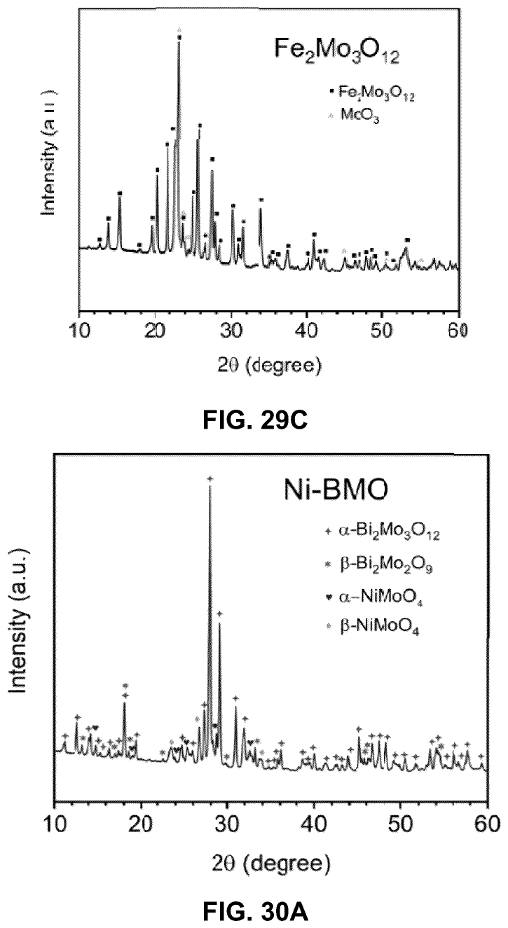

FIGS. 29A-29C show XRD of referenced mixed MoOx detected in the disclosed catalyst formulations.

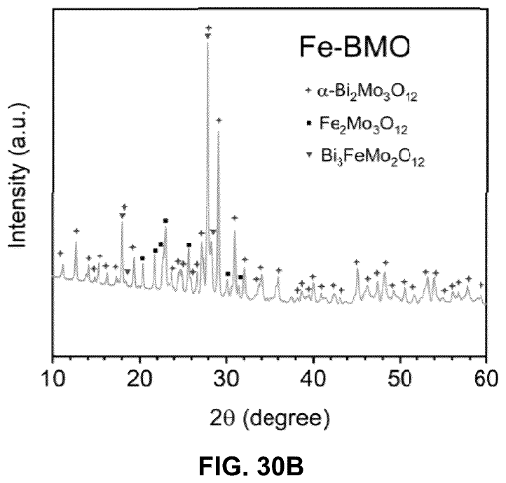

FIGS. 30A-30B show XRD of Ni-BMO and Fe-BMO, respectively, demonstrating presence of α-BMO in Fe-BMO. See phases detected in various catalysts in Table 4 below:

TABLE 4
Phases Detected in Various Catalysts

| Catalyst | Phase(s) Detected by XRD |
|---|---|
| Bi₂Mo₃O₁₂ | α-Bi₂Mo₃O₁₂ |
| NiMoO₄ | α-NiMoO₄, β-NiMoO₄ |
| Fe₂MoO₁₂ | Fe₂Mo₃O₁₂, MoO₃ |
| NiBiMo₃Oₓ | α-Bi₂Mo₃O₁₂, β-Bi₂Mo₂O₉, α-NiMoO₄, β-NiMoO₄ |
| FeBiMo₃O₃ | α-Bi₂Mo₃O₁₂, Fe₂Mo₃O₁₂, Bi₃FeMo₂O₁₂ |
| Ni₁₁BiMo₁₂Oₓ | α-Bi₂Mo₃O₁₂, α-NiMoO₄, β-NiMoO₄ |
| Ni_{10.5}Fe_{0.5}BiMo₁₂Oₓ | α-Bi₂Mo₃O₁₂, α-NiMoO₄, β-NiMoO₄ |
| Ni₁₀FeBiMo₁₂Oₓ | α-Bi₂Mo₃O₁₂, FeMoO₄, Fe₂Mo₃O₁₂, α-NiMoO₄, β-NiMoO₄ |
| Ni₈Fe₃BiMo₁₂Oₓ | α-Bi₂Mo₃O₁₂, FeMoO₄, Fe₂Mo₃O₁₂, α-NiMoO₄, β-NiMoO₄ |

### Example 8: Performance Under FDO Modes

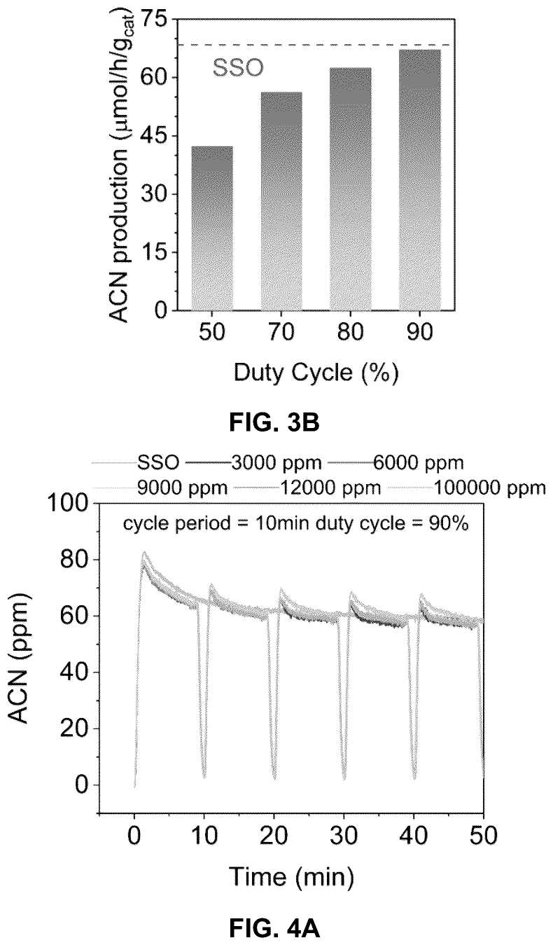

With the information gained from the temperature programmed reactions, FDO testing was done with the Ni₈Fe₃-BMO at 330° C., where the ACN yield is decent and the NH₃ conversion has not reached 100%, at which point the ACN selectivity will be limited. The FDO evaluations were begun with a normal feed-O₂ regen FDO scheme described in Table 5, with varied duty cycle from 50% to 90%. In this FDO scheme, the ACN will only be produced in phase 1. Since the feed conditions are the same as SSO, it is referred to as normal feed phase. In phase 2, the C₃H₆ and NH₃ were cut-off, only O₂ was present. In FIG. 3A, the ACN transient response is shown for FDO and SSO. The FDO response exhibits an overshoot of ACN when phase 2 is transitioning to phase 1, and then decreases. The sharp rise is referred to as an ACN “spike”. The ACN spikes were usually above the SSO trace, meaning under this FDO mode, a portion of the ACN formation rate has exceeded that of SSO. Although phase 2 does not produce ACN, its function serves as a regenerative phase, this is referred to as the O₂ regen phase. Shown in FIG. 3B is the ACN production for FDO and SSO calculated by averaging the performance in the first 80 min. As the duty cycle is increased, the ACN production also increases, at 90% duty cycle, the FDO performance is close to that of the SSO.

TABLE 5
Gas concentrations in the normal feed-O₂ regen FDO scheme

| Phase 1 | | | Phase 2 | | |
|---|---|---|---|---|---|
| C₃H₆ | NH₃ | O₂ | C₃H₆ | NH₃ | O₂ |
| 1500 ppm | 1500 ppm | 3000 ppm | 0 | 0 | 3000 ppm |

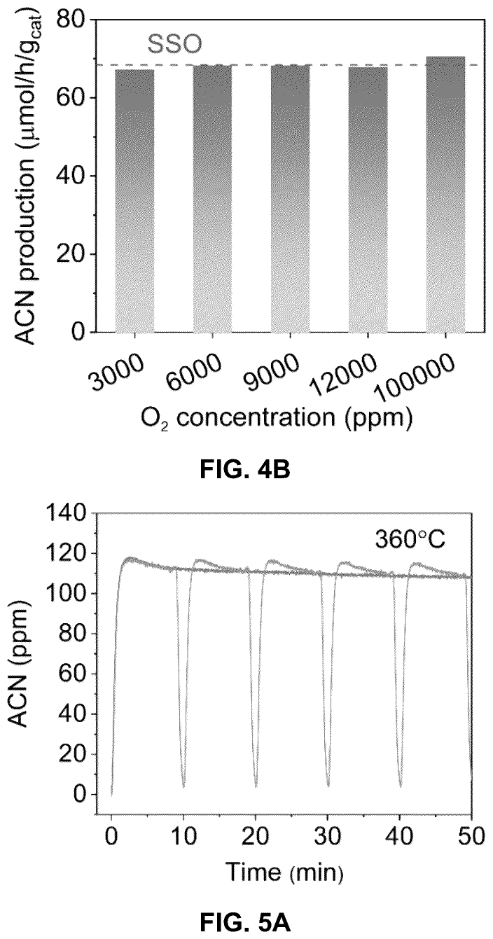

The ACN productivity can be further increased by increasing the O₂ concentration in the regeneration phase. Shown in FIG. 4A, at 330° C., cycle period=10 min and the duty cycle fixed at 90%, the O₂ concentration in phase 2 is increased from 0.3% to 10% ppm. The ACN production increased with O₂ concentration. At O₂=10% ppm, the FDO performance exceeds that of the SSO, as can be seen in FIG. 4B.

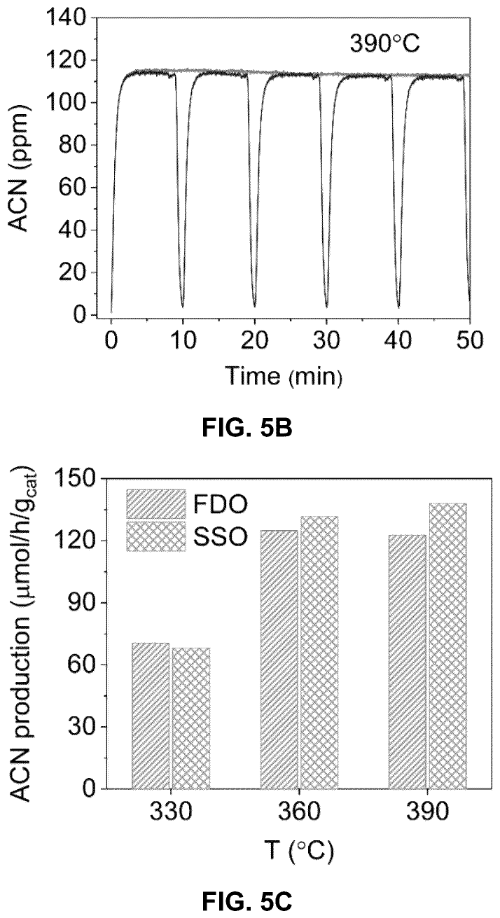

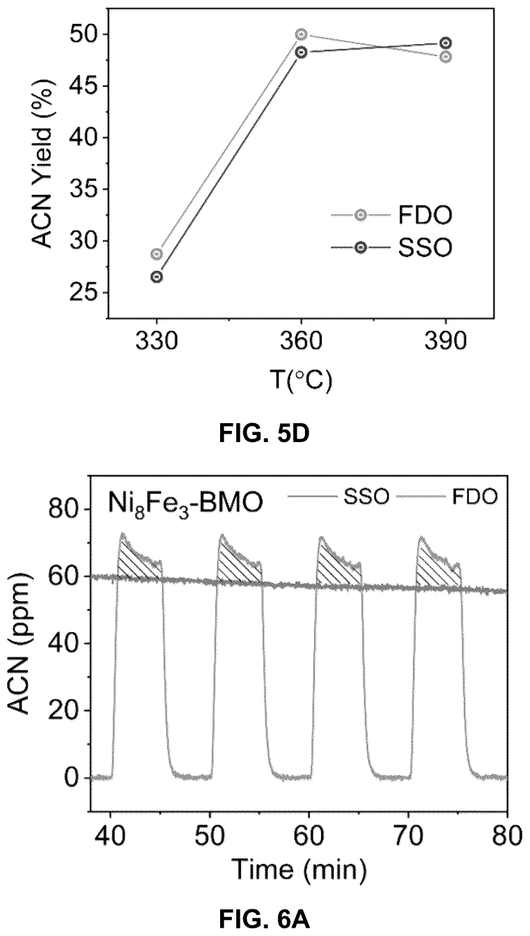

The best FDO scheme above with elevated O₂ input in the O₂ regen phase (O₂=10%) was applied at higher temperatures, 360° C. and 390° C. (FIGS. 5A-5B). The ACN spikes are less prominent at elevated temperatures. The advantages in FDO mode in ACN production is decreased at 360° C. and 390° C. (FIG. 5C). Nonetheless, in comparison to SSO, FDO still shows higher performance up to 360° C. in terms of ACN yield (FIG. 5D). Ammonia burn (the preferential oxidation of NH₃ to N₂) probably hampers the potential for ACN production increase at elevated temperatures.

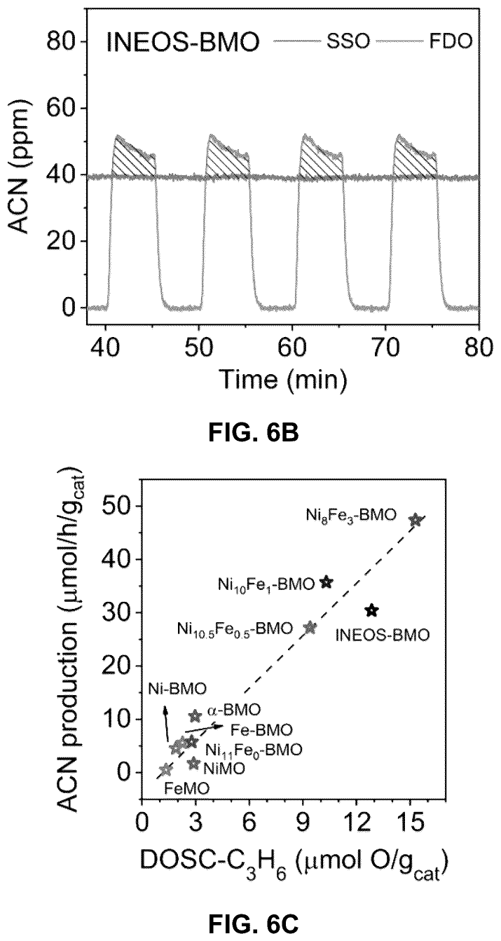

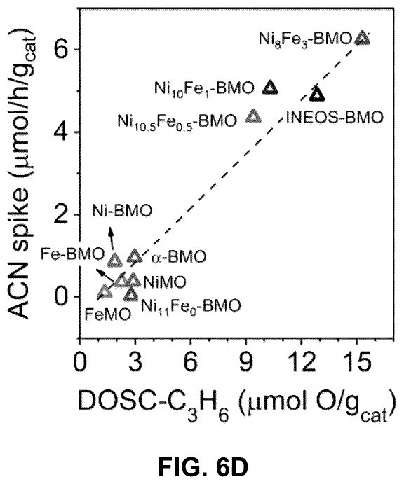

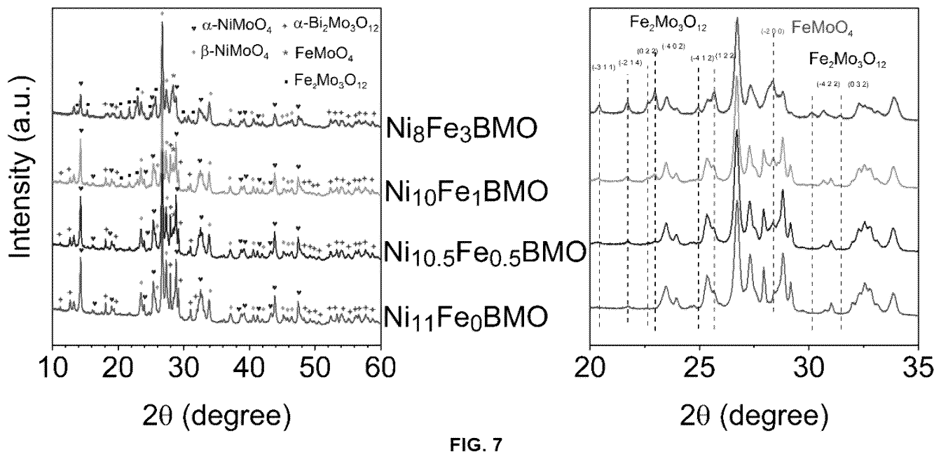

The overshoot phenomenon of a desired product in FDO schemes that rely on O₂ regeneration is believed to be related to lattice oxygen on/in the catalyst in certain partial oxidation reactions. The area where the ACN trace in FDO is higher than the SSO is defined as the ACN spike region, using Ni₈Fe₃-BMO as an example shown in FIG. 6A (shaded area). The FDO conditions used are the same as those described in Table 5 with a cycle period=10 min and duty cycle of 50% at 330° C. The ACN production and the production associated within the ACN spike region using this FDO condition are used as indicators to evaluate the performance of various BMO-based catalysts and transition metal-doped molybdate reference materials (FIGS. 6A-6B). The effect of lattice oxygen by dynamic oxygen storage capacity (DOSC) measurements was further investigated using C₃H₆ as a probe agent. A near linear correlation was found between DOSC and ACN production and ACN spike in FDO amongst the transition metal promoted BMO-based catalysts. As seen in FIG. 6C-D ACN production increases with DOSC. Furthermore, the promoters Ni and Fe must both be present in the BMO catalyst formulation to observe the promotional effect, and the Ni/Fe ratio to plays a role in the promotion of ACN productivity where with increasing Fe doping the performance is increased. Here, Ni:Fe=8:3 shows the best catalytic performance. XRD patterns for NiFe-BMO show the evolution of Fe₂Mo₃O₁₂ and FeMoO₄ growth with increasing Fe doping from 0 to 3 (FIG. 7). It is noted that Fe²⁺ containing FeMoO₄ was never detected in FeMO or Fe-BMO catalysts, it only emerged in NiFe-BMO samples, Ni might be involved in Fe²⁺ formation. It was previously found that the cooperation of Fe³⁺⁻containing α-BMO phase and Fe²⁺-molybdate phase (stabilized by Ni or Co) is crucial to the ammoxidation performance in that the first is a catalytically active phase and the second one is the auxiliary re-oxidation phase. The good linearity between DOSC and ACN productions on NiFe-BMOs further proves that the oxygen mobility is enhanced by Fe²⁺ and Fe³⁺ oxide species in BMO and in turn boosts the ACN yield. The present disclosure utilized FDO to amplify these advantages in NiFe promoted BMO catalysts.

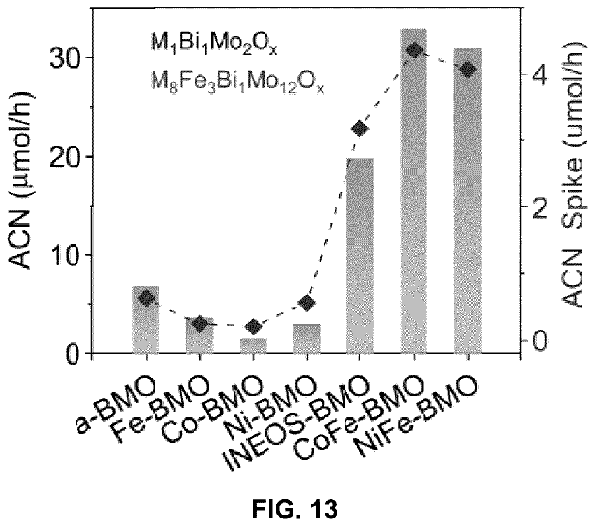

FIG. 13 shows acrylonitrile production in μmol per hour with different BMO-containing catalysts doped with transition metals including iron, cobalt, and nickel, as well as cobalt with nickel. Both CoFe-BMO and NiFe-BMO exhibit good acrylonitrile productivity in FDO.

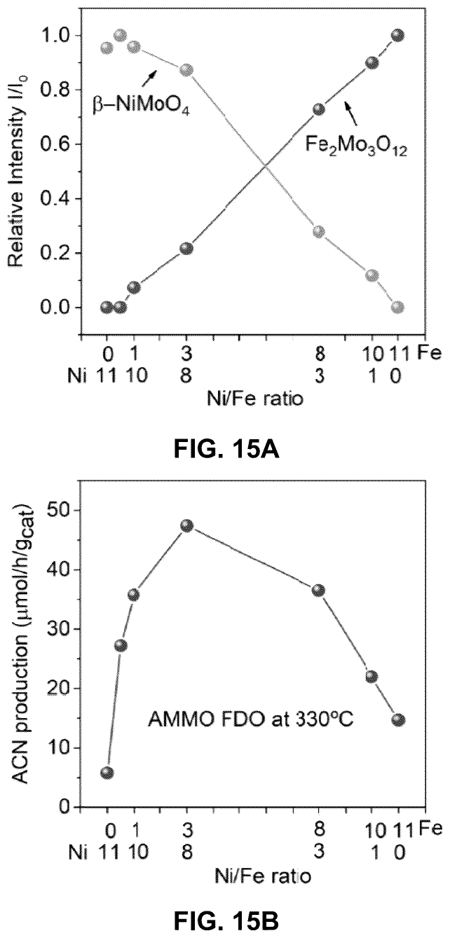

FIG. 15A shows effect of Ni/Fe ratio in NiFe-BMO using fresh catalyst. FIG. 15B is a graph showing steady-state ACN production on the NiFe-BMO catalyst with varied O₂ concentration at 330° C. Ni₈Fe₃ BMO shows the best acrylonitrile production in FDO of materials tested. The reaction temperature was 330° C. The catalyst was first treated with 10% O₂/N₂ for 1 h and then FDO was begun. Cycling was between C₃H₆:NH₃:O₂=0.15%:0.15%:0.3% and O₂=3% with a cycle period of 10 minutes and a 50% duty cycle.

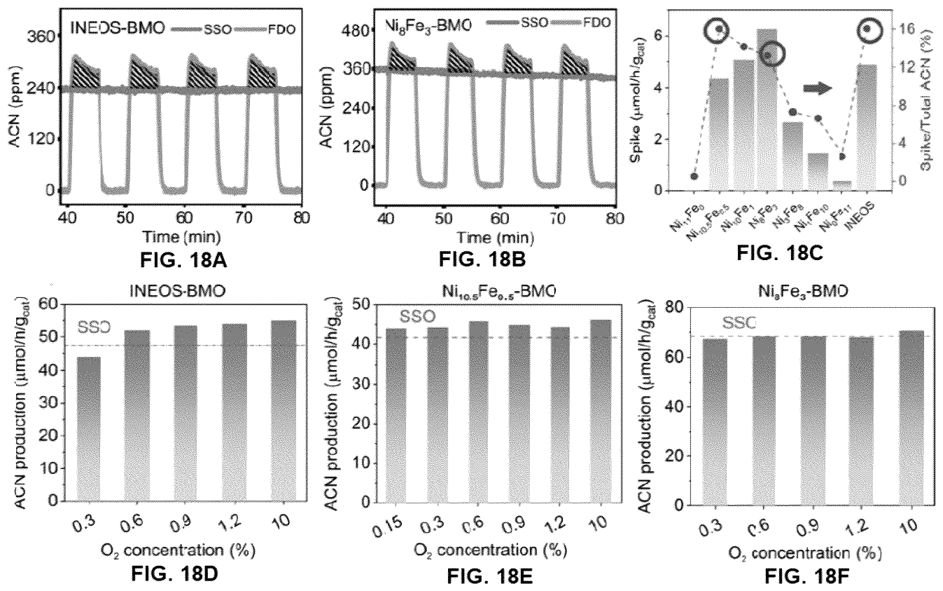

FIGS. 18A-18F show the effect of Ni/Fe ratio in NiFe-BMO. Spike features (see shaded areas in FIGS. 18A-18B) decrease as Ni content decreases. Ni-rich formulations perform as well as or better than a commercial catalyst (INEOS).

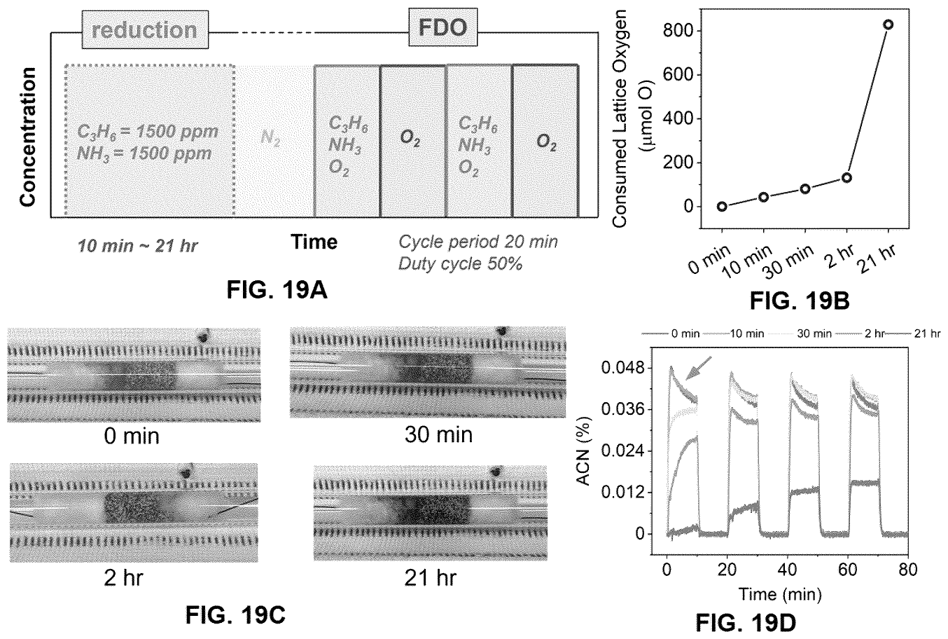

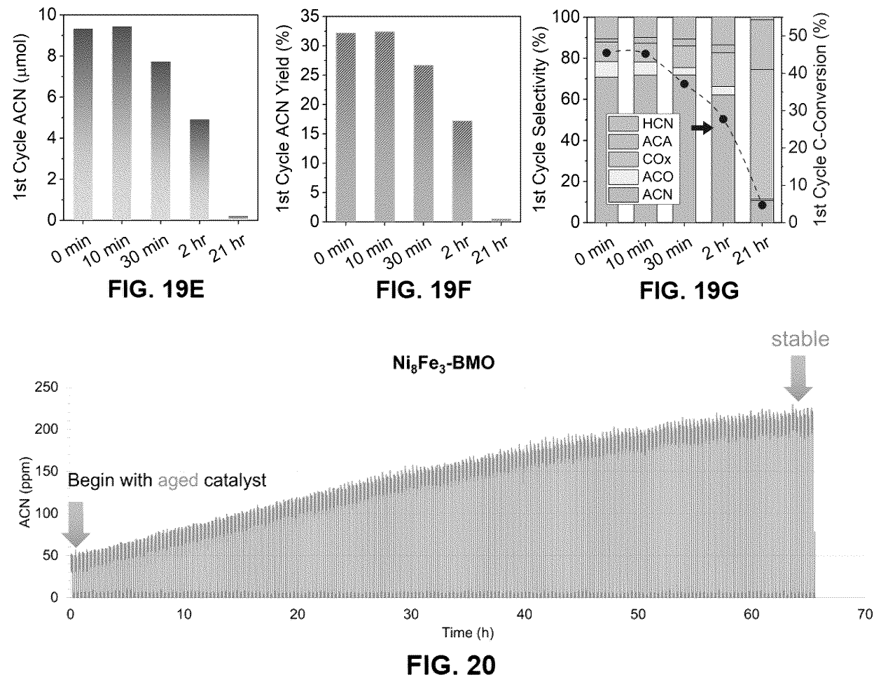

FIGS. 19A-19G show the effects of lattice oxygen availability. For reduction with C₃H₆ (1500 ppm)+NH₃ (1500 ppm), from 10-30 minutes, less than 1% lattice oxygen is removed, but after 21 hours, 8.4% lattice oxygen is removed. Decreased lattice oxygen results in decreased ACN activity and increased selectivity to C1/C2 products is observed. The experiment included 0.65 g of Ni₈Fe₃BMO and 1 g of SiO₂, with a total flow rate of 50 sccm, and the reaction temperature was 330° C.

FIG. 20 shows the effects of catalyst aging. The Ni₈Fe₃-BMO catalyst was aged with C₃H₆:NH₃:O₂=0.15%:0.15%:0.15% for 3 days and FDO was then begun. Cycling was carried out between C₃H₆:NH₃:O₂=0.15%:0.15%:0.15% and O₂=0.3% with a cycle period of 20 min and a 50% duty cycle.

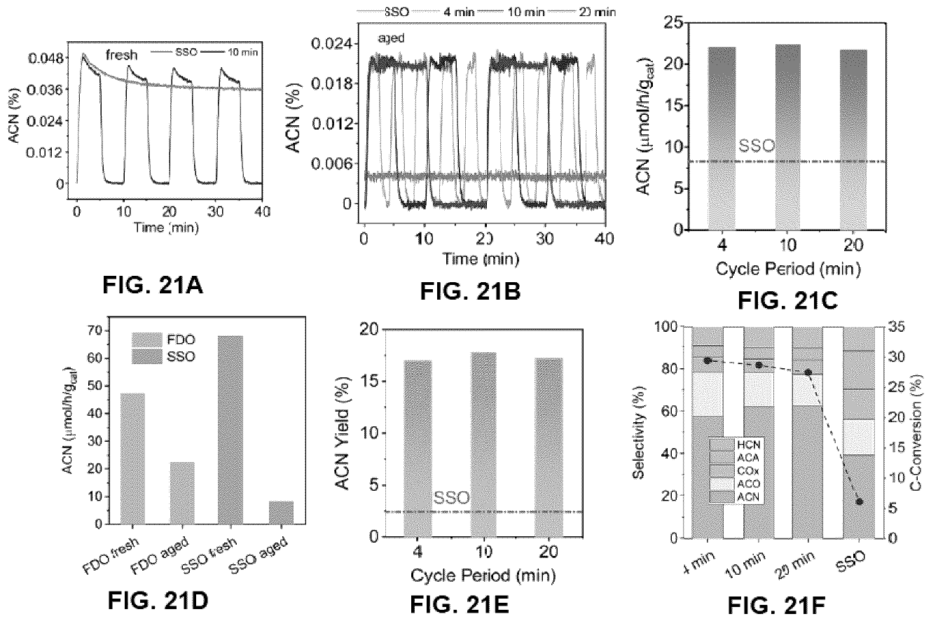

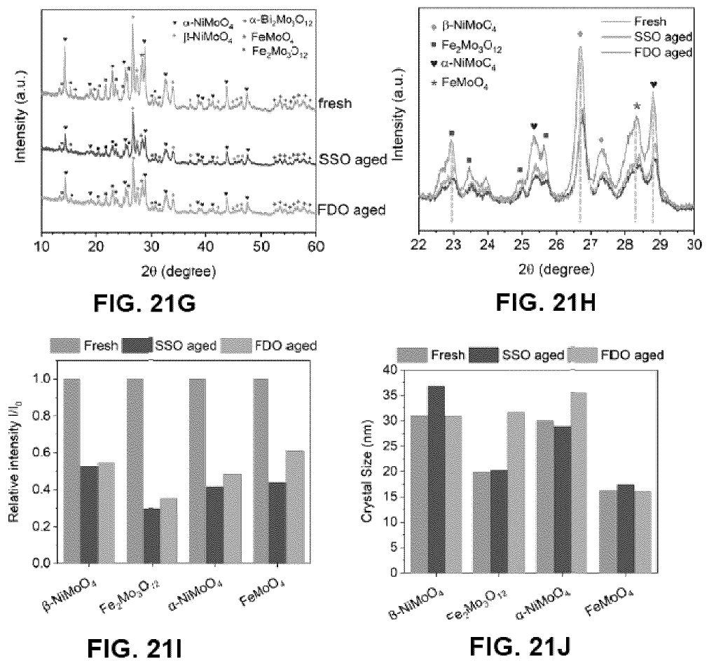

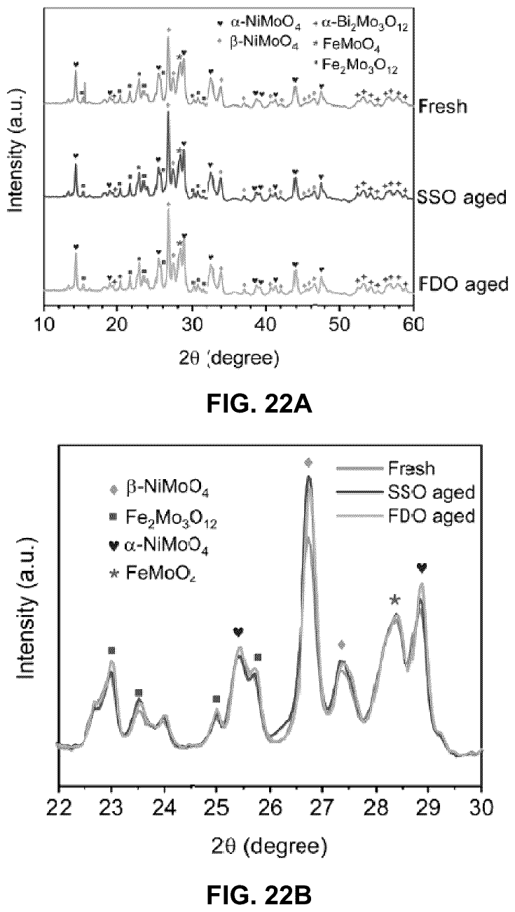

FIGS. 21A-21J show further effects of catalyst aging. For SSO fresh and FDO fresh, the samples were pretreated in 10% O₂ at 500° C. for 1 h. For SSO aged, the sample was aged in SSO conditions for 3 days before measurement. For FDO aged, the sample was first aged in SSO conditions for 3 days then FDO conditions for 65 h. Although Ni₈Fe₃-BMO performance decreased after aging in general, FDO aged sample out-perform SSO aged one in ACN production and yield in every cycle period from 4-20 min with just 50% duty cycle, O₂=3000 ppm in phase 2. In comparison, at the fresh state, the need for duty cycle and O₂ input in phase 2 are more intense for FDO to be able to out-perform SSO as shown in FIG. 18F. In FIGS. 21G-21J, the SSO aged and FDO aged samples were pelletized for catalytic performance evaluation purpose. In contrast, the fresh catalyst was not pelletized. FIGS. 21G-21J and FIGS. 22A-22B show structural changes in aged catalysts. Note the SSO aged and FDO aged samples were not pelletized in FIGS. 22A-22B for more a rigorous comparison with the fresh sample. For the pelletized scenario, all phases decrease in intensity after reaction and they appear to undergo larger changes in SSO versus FDO. For the not-pelletized scenario, the phase change was negligible after SSO or FDO. This could indicate that the pelletized catalyst provide a more thorough interaction between feed gas and the catalyst particles.

SSO was conducted under C₃H₆:NH₃:O₂=1500:1500:3000 ppm at 330° C. for 3 days. FDO cycling was carried out between C₃H₆:NH₃:O₂=1500:1500:3000 ppm and O₂=3000 ppm at 330° C. for 3 d.

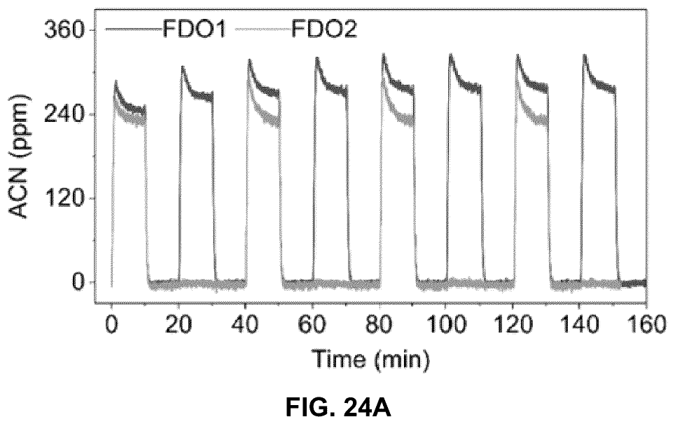

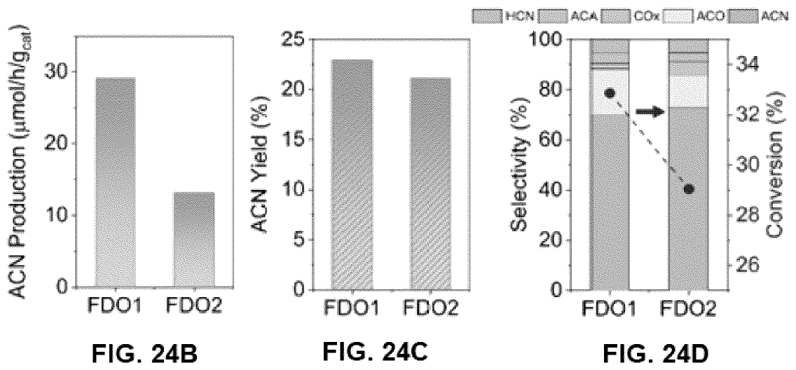

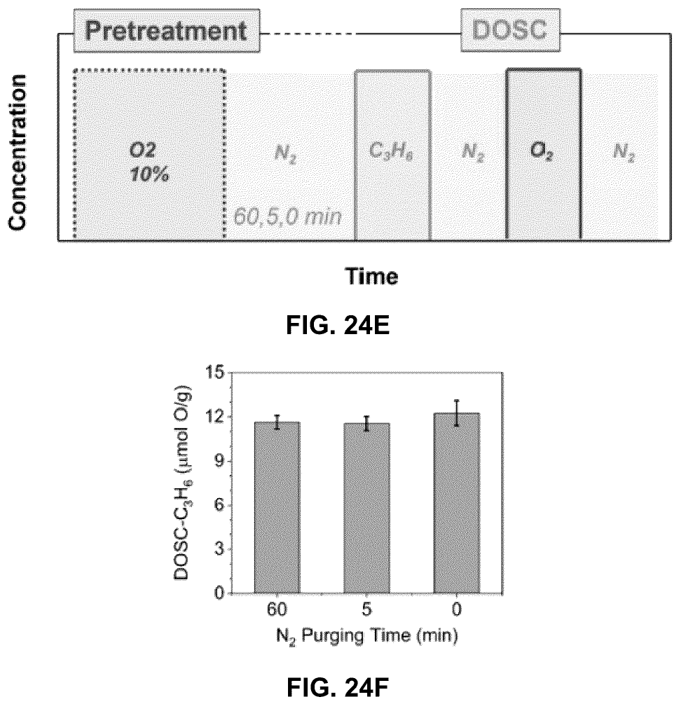

FIGS. 24A-24F show the effect of N₂ flushing to evaluate the effect of chemisorbed oxygen (or so-called surface oxygen). The total flow rate was 50 sccm at a temperature of 330° C. For FDO1, C₃H₆:NH₃:O₂=1500:1500:3000 ppm for 10 minutes cycled with O₂ at 3000 ppm for 10 minutes. For FDO 2, C₃H₆:NH₃:O₂=1500:1500:3000 ppm for 10 minutes cycled with N₂ for 10 minutes, O₂ at 3000 ppm for 10 minutes, and N₂ for 10 minutes. Essentially FDO2 adds an N₂ flushing phase between the reaction phase and the regeneration phase, enlarging the cycle period from 20 min to 40 min, thus cutting the ACN production by 50% compared to FDO1, but the ACN yield, selectivity and C-conversion is not significantly changed. DOSC conducted with different N₂ purging time utilized a protocol that pretreats the sample in 10% O₂ for 1 h at 330° C. then purged with N₂ for 60 min, 5 min or 0 min. The results show that with N₂ purging the DOSC decreased a little but within error range. The above catalytic performance result combined with DOSC results show that the effluence of chemisorbed oxygen does not affect the FDO performance much, it was the lattice oxygen (under catalyst surface) that play a more important role.

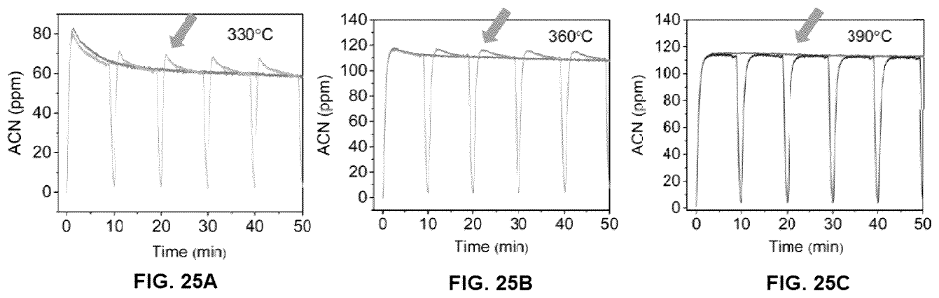

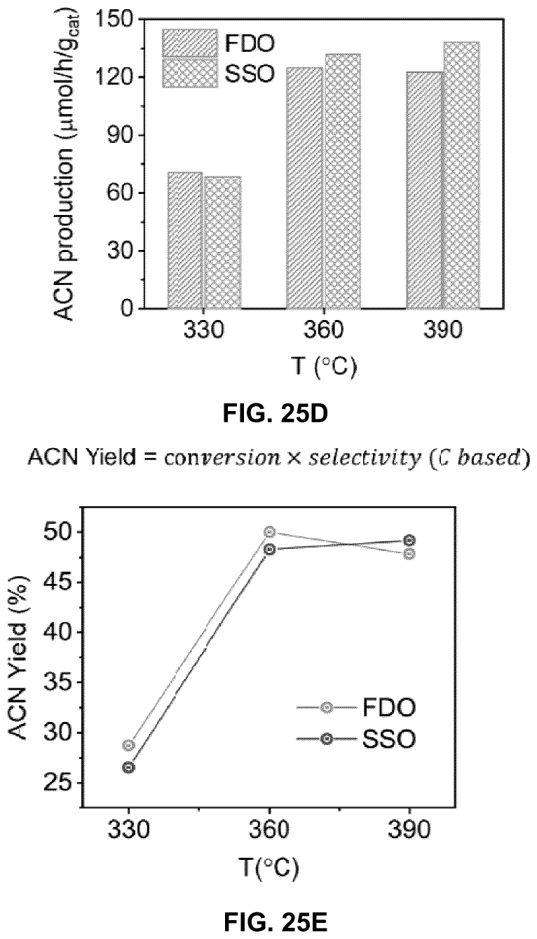

FIGS. 25A-25E show the effect of temperature changes. The acrylonitrile spike (area above the solid line) becomes weaker at higher temperatures. Phase 1 was C₃H₆:NH₃:O₂=1500:1500:3000 ppm, phase 2 was 10% O₂, with a cycle period of 10 minutes at 90% duty cycle.

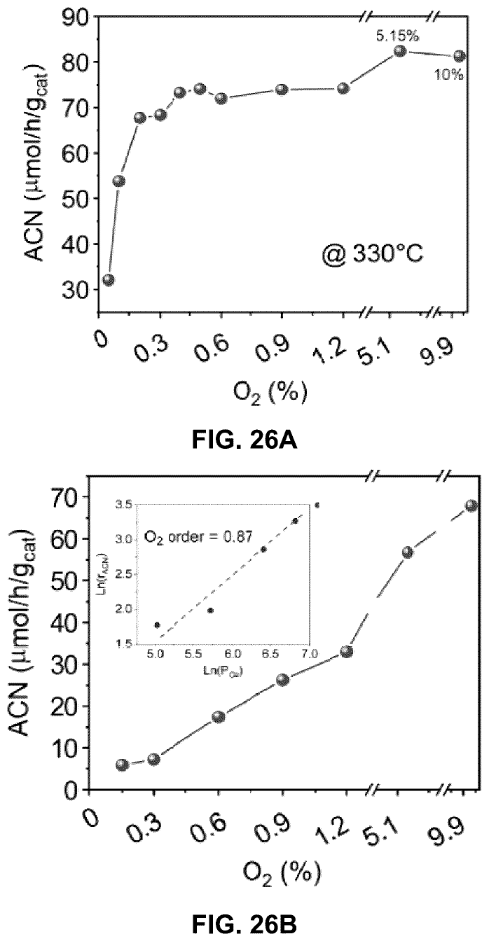

FIGS. 26A-26B show the O₂ reaction order for fresh (FIG. 26A) and aged (FIG. 26B) catalyst. FDO does not show an improvement in ACN yield at high temperatures. Oxygen concentrations varied from 0.05% to 10%, with a 50 sccm flow rate, with feed gas conditions of 0.15% C₃H₆ and 0.15% ammonia, with the balance being N₂, with T=330° C. Fresh: data points collected in separate runs; before each run the sample was pre-treated with 10% O₂ in N₂ at 500° C. for 1 h. Aged: data points were collected in one continuous run, with the catalyst being treated in C₃H₆:NH₃:O₂=0.15%:0.15%:0.15% for 2 days before measurement.

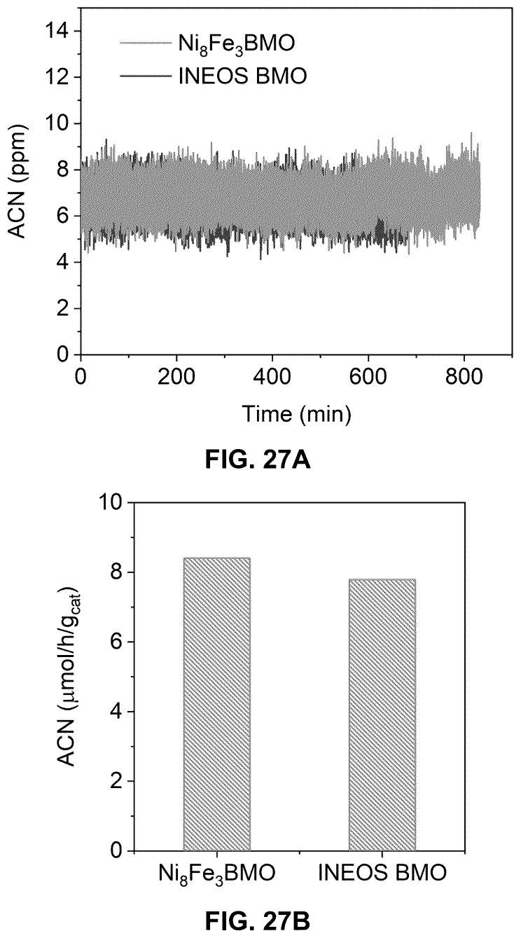

FIGS. 27A-27B show the effect of aging on the disclosed catalysts relative to a commercial catalyst. The catalyst was aged at 330° C. with C₃H₆:NH₃:O₂=1500:1500:3000 ppm for 3 days before measurement.

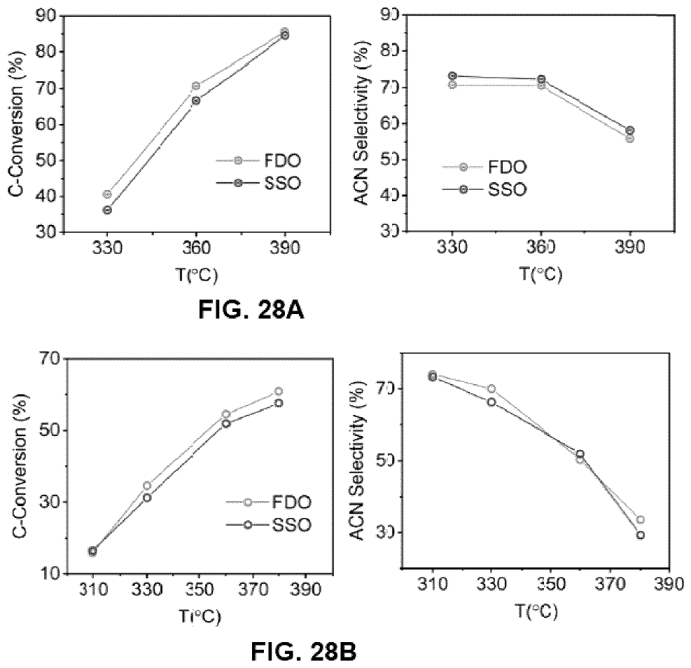

FIGS. 28A-28B show the effect of temperature on the disclosed catalysts (FIG. 28A) relative to a commercial catalyst (FIG. 28B).

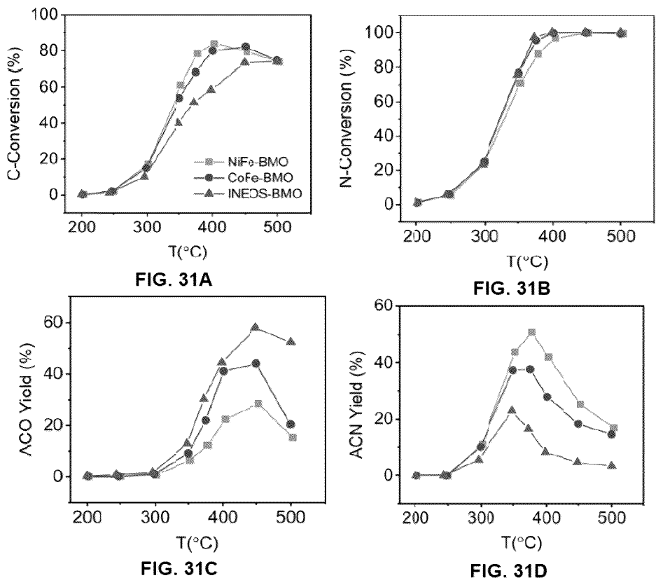

FIGS. 31A-31D show, respectively, C conversion, N conversion, ACO yield, and ACN yield for NiFe-BMO and CoFe-BMO relative to a commercial catalyst sample.

### Example 9: Processes Leveraging Excess O₂

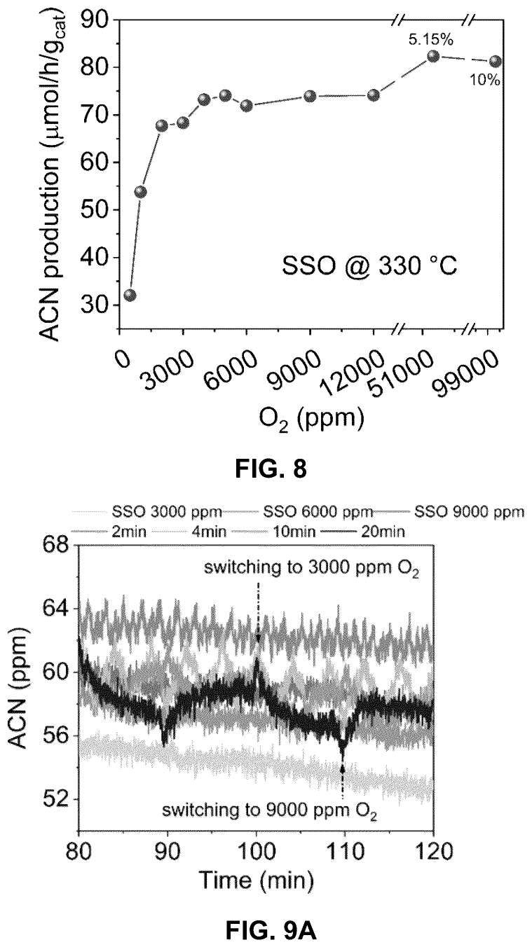

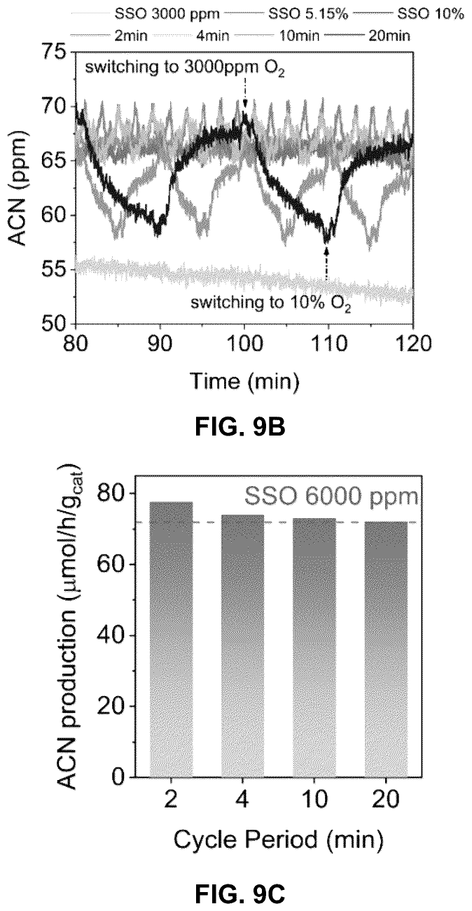

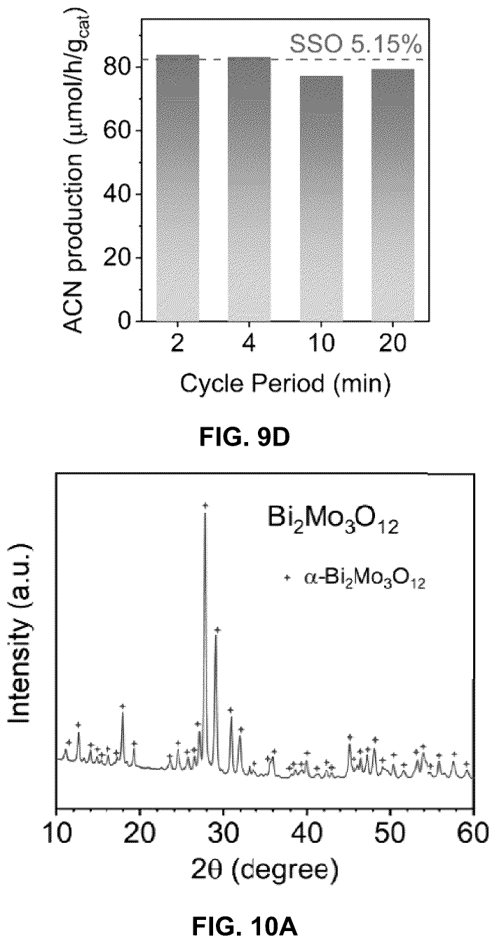

To further explore the disclosed processes in relation to FDO, a new strategy was introduced, the feed gas conditions are listed in Table 6. Here two schemes that leverage a high concentration O₂ were proposed where C₃H₆ and NH₃ are not cut-off in phase 2. The impact of O₂ concentration in SSO on the Ni₈Fe₃-BMO was investigated first at 330° C. The O₂ concentration is varied from 500 ppm to 10%, with C₃H₆ and NH₃ fixed to 1500 ppm, total flowrate=50 sccm, C₃H₆ GHSV=0.65 h⁻¹. The investigation was done with separate runs, each run with an increased O₂ concentration. The catalyst was pretreated with 10% O₂/N₂ before every run, each run lasts ˜4 h, the ACN productivity was recorded after 2 h. From the ammoxidation reaction, the stoichiometric ratio of O₂/C₃ is 1.5:1, hence 2250 ppm O₂ should be supplied with 1500 ppm C₃H₆, theoretically. But in practice O₂/C₃ is usually in slight excess (2:1). The results in FIG. 8 show that ACN production increased with O₂ concentration and reaches a plateau around O₂/C₃=1.5:1, right around the stoichiometric point. The ACN productivity increased ˜10% from 70 μmol/h/g_{cat}, to 80 μmol/h/g_{cat} when the O₂ is in the 5% to 10% range (well above the stoichiometric point). The results reveal that with high O₂ concentration, the catalyst performs better. In the following FDO schemes, the C₃H₆ and NH₃ concentrations are fixed to 1500 ppm, and the O₂ concentration is periodically changed from 3000 ppm to 9000 ppm (scheme 1) or 10% (scheme 2). FIGS. 9A-9B and FIGS. 9C-9D illustrated the ACN traces for the FDO experiment and the associated ACN production for schemes 1 and 2 respectively. In scheme 1, it was shown that the ACN production monotonically increased with decreased cycle period. With the average O₂ concentration being 6000 ppm in this scheme, the SSO at O₂ 6000 ppm was used as a benchmark. FDO with a cycle period of 2˜4 min shows higher ACN production than SSO. A similar trend was also seen with Scheme 2, with FDO performance with a cycle period 2˜4 min exceeding that of SSO with an average O₂ concentration 5.15%.

TABLE 6
FDO schemes leveraging excess O₂

| Cycling | Phase 1 | | | Phase 2 | | |
|---|---|---|---|---|---|---|
| scheme | C₃H₆ | NH₃ | O₂ | C₃H₆ | NH₃ | O₂ |
| Scheme 1 | 1500 ppm | 1500 ppm | 3000 ppm | 1500 ppm | 1500 ppm | 9000 ppm |
| Scheme 2 | 1500 ppm | 1500 ppm | 3000 ppm | 1500 ppm | 1500 ppm | 10% |

Cycle period = 2, 4, 10, 20 min, duty cycle = 50%, T = 330° C.

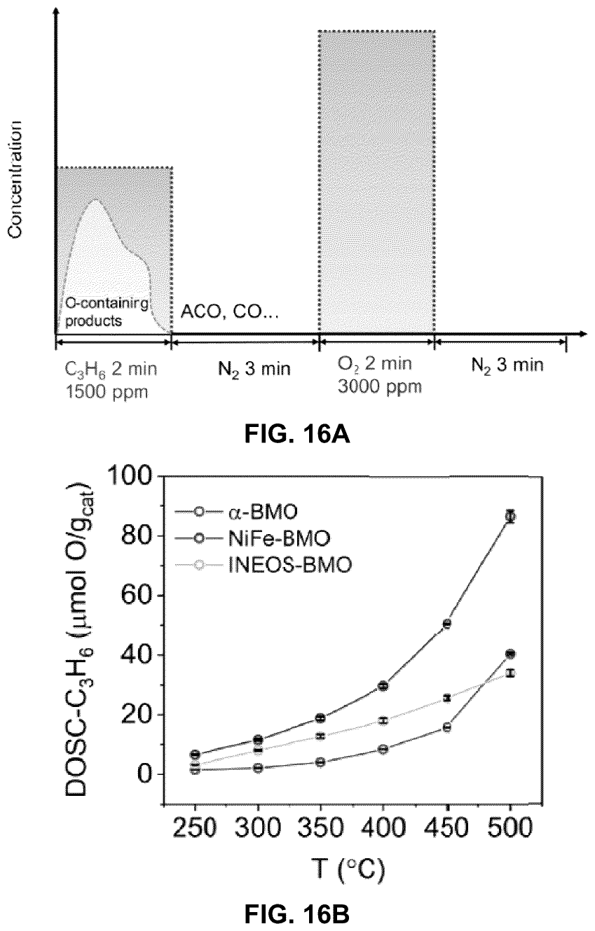

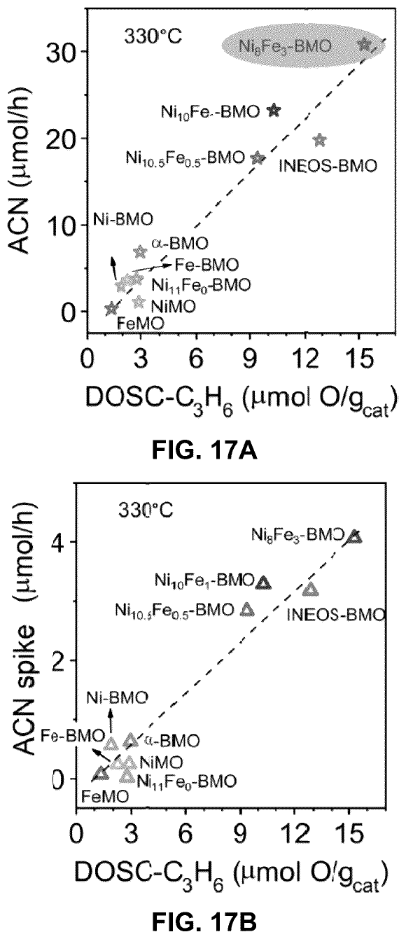

FIGS. 16A-16B show dynamic oxygen storage capacity (DOSC) for C₃H₆ using different BMO catalysts. FIGS. 17A-17B show DOSC-C₃H₆ versus FDO ACN production and ACN spike for different BMO catalysts. DOSC shows a relationship to ACN production of the catalysts in FDO, with FDO performance being tunable by varying the Ni/Fe ratio.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Becker, T. et al. 2021. “Decentralized Modular Production to Increase Supply Chain Efficiency in Chemical Markets: An Example of Polymer Production.” Journal of Business Economics 91 (6): 867-95.
2. Brazdil, J. F. 2018. “Designing Multifunctionality into Single Phase and Multiphase Metal-Oxide-Selective Propylene Ammoxidation Catalysts.” Catalysts 8 (3): 103.
3. Callahan, J.L. et al. 1970. “Oxidation and Ammoxidation of Propylene over Bismuth Molybdate Catalyst.” Industrial and Engineering Chemistry Product Research and Development 9 (2): 134-42.
4. Chang, T.S. et al. 2000. “Catalytic Behavior of BiPO4 in the Multicomponent Bismuth Phosphate System on the Propylene Ammoxidation.” Catalysis Letters 68 (3-4): 229-34.
5. Fouladvand, S. et al. 2016. “Unsteady-State Operation of Supported Platinum Catalysts for High Conversion of Methane.” Chemical Engineering Journal 292: 321-25.
6. Grasselli, R.K. 2011. “Chapter 5. Ammoxidation of Propylene and Propane to Acrylonitrile.” In Nanostructured Catalysts: Selective Oxidations, 96-140. The Royal Society of Chemistry.
7. Huang, X. F., et al. 2001. “Investigation of the Unsteady-State Oxidation of n-Butane to Maleic Anhydride in Fixed-Bed Reactors.” Industrial and Engineering Chemistry Research 40 (3): 768-73.
8. Jain, A K, et al. 1983. “Influence of Forced Feed Composition Cycling on the Rate of Ammonia Synthesis over an Industrial Iron Catalyst Part I—Effect of Cycling Parameters and Mean Composition.” The Canadian Journal of Chemical Engineering 61 (6): 824-32.
9. Lang, X. et al. 1989. “Maleic Anhydride Formation from 1, 3-butadiene Oxidation over a Vanadia Molybdate Catalyst.” The Canadian Journal of Chemical Engineering 67 (3): 423-31.
10. Licht, R.B. et al. 2016. “The Mechanism and Kinetics of Propene Ammoxidation over α-Bismuth Molybdate.” Journal of Catalysis 339: 228-41.
11. Silveston, P.L., et al. 2007. Periodic Operation of Reactors. Cyclic Separating Reactors. doi.org/10.1002/9780470988688.ch2.
12. Idol, Jr., J.D. 1959. U.S. Pat. No. 2,904,580.
13. Suresh, D.D. et al. 1992. U.S. Pat. No. 5,093,299.
14. Guan, X. et al. 2003. U.S. Pat. No. 6,596,897.
15. Paparizos, C. et al., 2006, U.S. Pat. No. 7,071,140.
16. Brazdil, J.F. et al., 2020, U.S. Pat. No. 10,780,427.

What is claimed is:

1. A method for manufacturing an acrylic compound or intermediate, the method comprising alternately flowing a first gas phase and a second gas phase over a catalyst in a reactor;
wherein the first gas phase, the second gas phase, or both comprise propene, ammonia, and oxygen;
wherein the catalyst comprises a transition metal promoted bismuth molybdate catalyst;
wherein at least one reactor input or condition is modified periodically using forced dynamic operation (FDO); and
wherein the acrylic compound or intermediate comprises acrylonitrile, acrolein, acrylic acid, or any combination thereof.

2. The method of claim 1, wherein the oxygen is provided as substantially pure oxygen gas.

3. The method of claim 1, wherein the oxygen is provided as a component of a mixed gas composition.

4. The method of claim 1, further comprising a third gas phase.

5. The method of claim 4, wherein the third gas phase comprises propene, ammonia, oxygen, or any combination thereof.

6. The method of claim 1, wherein the at least one reactor input comprises composition of the first gas phase, composition of the second gas phase, flow rate of the first gas phase, flow rate of the second gas phase, duty cycle of the reactor, reaction temperature, cycle period of the reactor, or any combination thereof.

7. The method of claim 1, wherein the first gas phase comprises propene, ammonia, and oxygen in a molar ratio of about 1:1:2.

8. The method of claim 1, wherein the second gas phase is selected from oxygen or a combination of propene, ammonia, and oxygen in a molar ratio of from about 1:1:6 to about 1:1:67.

9. The method of claim 8, wherein the second gas phase is oxygen and wherein the oxygen is present in a molar amount of from about 0.5 to about 67 times a molar amount of propene in the first gas phase.

10. The method of claim 1, wherein the first gas phase, the second gas phase, and, if present, the third gas phase, or any combination thereof further comprise an inert gas.

11. The method of claim 6, wherein the cycle period of the reactor is from about 0.1 min to about 2 hours.

12. The method of claim 6, wherein the duty cycle of the reactor is from about 50% to about 95%.

13. The method of claim 6, wherein the reaction temperature is from about 300° C. to about 500° C.

14. The method of claim 1, wherein the transition metal promoted bismuth molybdate catalyst has a formula MₐFe_{b}Bi_{c}Mo₁₂Oₓ, wherein M comprises a transition metal, an alkaline earth metal, an alkali metal, or any combination thereof;
wherein 0<a+b≤11;
wherein 0<c<3; and
wherein x has a value such that an amount of oxygen in the catalyst balances a charge of M, Fe, Bi, and Mo combined.

15. The method of claim 14, wherein a is from 0.1 to 10.9 and b is from 0.1 to 10.9

16. The method of claim 14, wherein M comprises Ni, Co, Mn, Mg, Zn, Cr, or any combination thereof.

17. The method of claim 14, wherein the catalyst comprises Ni₁₁Fe₀Bi₁Mo₁₂Oₓ, Ni_{10.5}Fe_{0.5}Bi₁Mo₁₂Oₓ, Ni₁₀Fe₁Bi₁Mo₁₂Oₓ, Ni₈Fe₃Bi₁Mo₁₂Oₓ, or any combination thereof.

18. The method of claim 1, wherein the catalyst further comprises a support material.

19. The method of claim 18 wherein the support material comprises silica.

20. The method of claim 18, wherein the support material is present at from about 20 wt % to about 80 wt % of the catalyst.
